(19) United States
(12) Patent Application Publication
(10) Pub. No.: US 2025/0120571 A1
(43) Pub. Date: Apr. 17, 2025

(54) ARTIFICIAL-INTELLIGENCE-BASED CONTROL SYSTEM FOR MECHANICALLY-ENHANCED INTERNAL IMAGING

(71) Applicant: SMART MEDICAL SYSTEMS LTD., Ra'Anana (IL)

(72) Inventor: Gad Terliuc, Ra'Anana (IL)

(21) Appl. No.: 18/688,608

(22) PCT Filed: Sep. 2, 2021
(86) PCT No.: PCT/US2021/071358

§ 371 Date: Mar. 1, 2024

(51) Int. Cl. A61B 1/00; A61M 25/10; G06T 7/00

(52) U.S. Cl. CPC A61B 1/00082; A61B 1/000094; A61B 1/000096; A61M 25/10184; G06T 7/0012; G06T 2200/24; G06T 2207/10068; G06T 2207/20081; G06T 2207/30004

(57) ABSTRACT

An artificial intelligence system is trained and used to detect procedure anomalies occurring during internal imaging procedures involving mechanically-enhanced or otherwise mechanically-altered tissue. An internal imaging device (e.g., endoscope) with a mechanical enhancement element alters tissue from its natural state or orientation such that regions of interest on the tissue may be more clearly distinguished from the surrounding tissue. Anomaly procedures associated with the alteration of the tissue and/or use of the internal imaging device can be detected by the artificial intelligence system, and remedial actions can be alerted and/or taken automatically.

### INCORPORATION BY REFERENCE

Reference is made to applicant's Published PCT Patent Applications WO2023/033859, WO2021/194872, WO2005/074377, WO2007/017854, WO2007/135665, WO2008/004228, WO2008/142685, WO2009/122395, WO2010/046891, WO2010/137025, WO2011/111040, and WO2012/120492, the disclosures of which are hereby incorporated by reference and made part of this specification.

### BACKGROUND

### Field

This disclosure relates generally to internal imaging, and more specifically to artificial-intelligence-based systems for internal image analysis and feedback.

### Description of the Related Art

Endoscopes generate imagery of internal body tissue. Health care professionals may use the images to identify regions of interest in the tissue, such as polyps or other lesions. To aid the health care professionals in identifying regions of interest, various aids such as artificial-intelligence-based image analysis systems may be employed.

### SUMMARY

The following presents a simplified summary of one or more aspects in order to provide a basic understanding of such aspects. This summary is not an extensive overview of all contemplated aspects, and is intended to neither identify key or critical elements of all aspects nor delineate the scope of any or all aspects. Its sole purpose is to present some concepts of one or more aspects in a simplified form as a prelude to the more detailed description that is presented later.

One aspect of the disclosure provides a system for machine-learning-based analysis of an endoscopy procedure. The system comprises: a balloon endoscope comprising a visualization element and an inflatable balloon, wherein the balloon endoscope is configured to mechanically enhance visualization of tissue when moved within an intestinal lumen of a patient with the inflatable balloon inflated to a sub-anchoring pressure, the inflatable balloon causing axial stretching of tissue of the intestinal lumen to at least partially flatten or unfold natural topography of the tissue; and a computing device comprising one or more processors and computer-readable memory. The computing device is programmed by executable instructions to at least: obtain an image of a portion of the tissue using the visualization element; analyze the image using a machine learning model trained to generate classification output data representing a procedure anomaly classification; determine, based at least partly on the classification output data, that the image corresponds to a procedure anomaly; generate feedback data based on the procedure anomaly, wherein the feedback data represents a remedial action to be taken with respect to the balloon endoscope; and send the feedback data to at least one of a user interface subsystem or a control subsystem.

The system of the preceding paragraph can in some embodiments include any of the following features, or any combination or sub-combination of the following features: wherein the computing device is programmed by further executable instructions to determine, based at least partly on the procedure anomaly being one of an under-inflation anomaly or an over-inflation anomaly, that the remedial action comprises a change to an inflation pressure of the balloon, and display, on a user interface, a message indicating a manner in which the inflation pressure is to be changed; wherein the computing device is programmed by further executable instructions to determine, based at least partly on the procedure anomaly being one of a low withdrawal speed anomaly or a high withdrawal speed anomaly, that the remedial action comprises a change to a withdrawal speed of the balloon endoscope, and display, on a user interface, a message indicating a manner in which the withdrawal speed is to be changed; wherein the procedure anomaly comprises a low withdrawal speed anomaly and the manner in which the withdrawal speed is to be changed comprises an increase in the withdrawal speed; wherein the procedure anomaly comprises a high withdrawal speed anomaly and the manner in which the withdrawal speed is to be changed comprises a reduction in the withdrawal speed; wherein the computing device is programmed by further executable instructions to determine, based at least partly on the procedure anomaly comprising detection of a tissue anomaly, that the remedial action comprises stopping withdrawal of the balloon endoscope, and display, on a user interface, a message indicating withdrawal of the balloon endoscope is to be stopped; wherein the computing device is programmed by further executable instructions to determine, based at least partly on the procedure anomaly comprising an under-inflation anomaly, that inflation of the balloon is to be confirmed, and display, on a user interface, a message indicating inflation of the balloon is to be confirmed; wherein the computing device is programmed by further executable instructions to determine, based at least partly on the procedure anomaly comprising a deflection rate anomaly, that the remedial action comprises a reduction in a deflection rate of the balloon endoscope, and display, on a user interface, a message indicating that the deflection rate of the balloon endoscope is to be reduced; wherein the computing device is programmed by further executable instructions to determine, based at least partly on the procedure anomaly being one of an over-inflation anomaly or a low withdrawal speed anomaly, that the remedial action comprises a reduction in an inflation pressure of the balloon, and generate a command for the control subsystem to reduce the inflation pressure of the balloon; wherein to reduce the inflation pressure of the balloon, the command causes the control subsystem to switch the inflation pressure of the balloon to a lower pressure level; wherein to reduce the inflation pressure of the balloon, the command causes the control subsystem to tune the inflation pressure of the balloon to a lower target pressure metric within a pressure range of a particular pressure level; wherein the computing device is programmed by further executable instructions to determine, based at least partly on the procedure anomaly being one of an under-inflation anomaly or a high withdrawal speed anomaly, that the remedial action comprises an increase in an inflation pressure of the balloon, and generate a command for the control subsystem to increase the inflation pressure of the balloon; wherein to increase the inflation pressure of the balloon, the command causes the control subsystem to switch the inflation pressure of the balloon to a higher pressure level; wherein to increase the inflation pressure of the balloon, the command causes the control subsystem to tune the inflation pressure of the balloon to a higher target pressure metric within a pressure range of a certain pressure level; wherein the computing device is programmed by further executable instructions to determine, based at least partly on the procedure anomaly comprising a tissue-anomaly-related procedure anomaly, that the remedial action comprises an increase in an inflation pressure of the balloon to an anchoring pressure, and generate a command for the control subsystem to increase the inflation pressure of the balloon to the anchoring pressure; wherein the tissue anomaly comprises at least one of a tumor, a serious bleeding, a perforation or tear in the intestinal tissue, and a serious adverse condition of the tissue; wherein to generate the command for the control subsystem, the computing device is programmed by further executable instructions to generate a command for a balloon inflation/deflation system; wherein the computing device is programmed by further executable instructions to obtain a second image using the visualization element, analyze the second image using the machine learning model to generate second classification output data, determine, based at least partly on the second classification output data, that the second image corresponds to no procedure anomaly, and cause presentation of a message regarding remediation of the procedure anomaly; a training computing device comprising one or more processors and computer-readable memory, the training computing device programmed by executable instructions to at least obtain a plurality of images, generate a plurality of training data images using the plurality of images, wherein images in a first subset of the plurality of training data images are associated with label data representing a negative classification for presence of the procedure anomaly, and wherein images in a second subset of the plurality of training data images are associated with label data representing a positive classification for presence of the procedure anomaly, train the machine learning model using the plurality of training data images, distribute the machine learning model to one or more endoscope systems; and wherein the procedure anomaly is one of a plurality of procedure anomalies that the computing device is configured to detect using the machine learning model.

Another aspect of the disclosure provides a computer-implemented method comprising, under control of a computer system comprising one or more processors configured to execute specific computer-executable instructions: obtaining an image of a portion of mechanically-enhanced tissue from an internal imaging device configured to mechanically enhance tissue; analyzing the image using a machine learning model trained to generate classification data representing a procedure anomaly classification; determining, based at least partly on the classification data, that the image corresponds to a procedure anomaly; generating feedback data based on the procedure anomaly, wherein the feedback data represents an action to be taken; and sending the feedback data to at least one of a user interface subsystem or a control subsystem associated with the internal imaging device.

The computer-implemented method of the preceding paragraph can in some embodiments include any of the following features, or any combination or sub-combination of the following features: wherein determining that the image corresponds to the procedure anomaly comprises determining that the image corresponds to one of an under-inflation anomaly associated with an inflatable balloon, an over-inflation anomaly associated with the inflatable balloon, a low retraction speed anomaly, a high retraction speed anomaly, or a deflection rate anomaly; determining the action to be taken based at least partly on the procedure anomaly, wherein the feedback data comprises a message to be displayed on a user interface regarding the action to be taken; determining the action to be taken based at least partly on the procedure anomaly, wherein the feedback data comprises a command for the control subsystem to perform the action to address the procedure anomaly; wherein determining the action to be taken comprises determining that the control subsystem is to perform at least one of reducing a degree of inflation of an inflatable balloon of the internal imaging device to a lower degree of sub-anchoring pressure, increasing a degree of inflation of an inflatable balloon of the internal imaging device to a higher degree of sub-anchoring pressure, increasing a degree of inflation of an inflatable balloon of the internal imaging device to an anchoring pressure, or deflating an inflatable balloon of the internal imaging device; obtaining a second image of a second portion of the mechanically-enhanced tissue from the internal imaging device, analyzing the second image using a second machine learning model trained to detect tissue anomalies, determining, based at least partly on results of analyzing the second image using the second machine learning model, that the second image corresponds to a tissue anomaly, and performing a remedial action comprising at least one of generating a command to the control subsystem to increase pressure of an inflatable balloon of the internal imaging device to an anchoring pressure, and displaying, on a user interface, a message instructing an operator to increase pressure of an inflatable balloon of the internal imaging device to an anchoring pressure; obtaining a second image of a second portion of the mechanically-enhanced tissue from the internal imaging device, analyzing the second image using a second machine learning model trained to detect tissue anomalies, determining, based at least partly on results of analyzing the second image using the second machine learning model, that the second image corresponds to a tissue anomaly, and performing a remedial action comprising at least one of generating a command to the control subsystem to switch a mechanical enhancement element of the internal imaging device to an anchoring state, and displaying, on a user interface, a message instructing an operator to switch a mechanical enhancement element of the internal imaging device to an anchoring state; wherein determining that the second image corresponds to the tissue anomaly comprises determining that the second image corresponds to at least one of: a tumor, a serious bleeding, a perforation or tear in tissue, and a serious adverse condition of tissue; obtaining a second image of a second portion of the mechanically-enhanced tissue from the internal imaging device, analyzing the second image using a second machine learning model trained to detect tissue anomalies, determining, based at least partly on results of analyzing the second image using the second machine learning model, that the second image corresponds to a tissue anomaly, performing a remedial action comprising at least one of generating a command to the control subsystem to deflate an inflatable balloon of the internal imaging device, and displaying, on a user interface, a message instructing an operator to deflate an inflatable balloon of the internal imaging device; and wherein determining that the second image corresponds to the tissue anomaly comprises determining that the second image corresponds to at least one of: a perforation of tissue, a major bleeding, and a serious adverse tissue condition.

A further aspect of the disclosure provides a system comprising: an internal imaging device comprising a visualization element and a mechanical enhancement element, wherein the mechanical enhancement element is configured to mechanically alter tissue, and wherein the visualization element is configured to generate images of mechanically-altered tissue; and a computing device comprising one or more processors and computer-readable memory. The computing device is programmed by executable instructions to at least: obtain an image of a portion of tissue using the visualization element; analyze the image using a machine learning model trained to generate classification output data representing a procedure anomaly classification; determine, based at least partly on the classification output data, that the image corresponds to a procedure anomaly; and generate feedback data based on the procedure anomaly, wherein the feedback data represents an action to be taken based on the procedure anomaly.

The system of the preceding paragraph can in some embodiments include any of the following features, or any combination or sub-combination of the following features: wherein the computing device is programmed by further executable instructions to determine the action to be taken, wherein the action to be taken comprises adjustment of an inflation pressure of the mechanical enhancement element based at least partly on the procedure anomaly being one of an under-inflation anomaly or an over-inflation anomaly, and wherein feedback data comprises a message, to be presented via a user interface, indicating a manner in which the inflation pressure is to be changed; wherein the computing device is programmed by further executable instructions to determine the action to be taken, wherein the action to be taken comprises adjustment of a speed of withdrawal of the internal imaging device based at least partly on the procedure anomaly being one of a low withdrawal speed anomaly or a high withdrawal speed anomaly, and wherein the feedback data comprises a message, to be presented via a user interface, indicating a manner in which the speed of withdrawal is to be adjusted; wherein the procedure anomaly comprises a low withdrawal speed anomaly and the manner in which the speed of withdrawal is to be adjusted comprises an increase in the speed of withdrawal; wherein the procedure anomaly comprises a high withdrawal speed anomaly and the manner in which the speed of withdrawal is to be adjusted comprises a reduction in the speed of the withdrawal; wherein the computing device is programmed by further executable instructions to determine the action to be taken, wherein the action to be taken comprises confirmation of inflation of the mechanical enhancement element, and wherein feedback data comprises a message, to be presented via a user interface, indicating that inflation of the mechanical enhancement element is to be confirmed; wherein the computing device is programmed by further executable instructions to determine the action to be taken, wherein the action to be taken comprises reduction in a deflection rate of the internal imaging device based at least partly on the procedure anomaly being a deflection rate anomaly, and wherein feedback data comprises a message, to be presented via a user interface, indicating a that the deflection rate of the internal imaging device is to be reduced; wherein to determine the action to be taken, the computing device is programmed by further executable instructions to determine that an inflation pressure of the mechanical enhancement element is to be reduced based at least partly on the procedure anomaly being one of an over-inflation anomaly or a low withdrawal speed anomaly, and wherein the feedback data comprises a command for a control subsystem to automatically reduce the inflation pressure of the mechanical enhancement element; wherein to determine the action to be taken, the computing device is programmed by further executable instructions to determine that an inflation pressure of the mechanical enhancement element is to be increased based at least partly on the procedure anomaly being one of an under-inflation anomaly or a high withdrawal speed anomaly, and wherein the feedback data comprises a command for a control subsystem to automatically increase the inflation pressure of the mechanical enhancement element; wherein to determine the action to be taken, the computing device is programmed by further executable instructions to determine that a pressure of the mechanical enhancement element is to be increased to an anchoring pressure based at least partly on the procedure anomaly comprising detection of a tissue anomaly, and wherein the feedback data comprises a command for a control subsystem to automatically increase the inflation pressure to the anchoring pressure; wherein to determine the action to be taken, the computing device is programmed by further executable instructions to determine that the mechanical enhancement element is to be switched to an anchoring state based at least partly on the procedure anomaly comprising detection of a tissue anomaly, and wherein the feedback data comprises a command for a control subsystem to automatically switch the mechanical enhancement element to the anchoring state; wherein to determine the action to be taken, the computing device is programmed by further executable instructions to determine, based at least partly on the procedure anomaly comprising a tissue-anomaly-related procedure anomaly, that the mechanical enhancement element is to be switched to an anchoring state and the internal imaging device is to be at least partially retracted, and wherein the feedback data comprises a message, to be presented via a user interface, indicating a that that the mechanical enhancement element is to be switched to the anchoring state and the internal imaging device is to be at least partially retracted; wherein the feedback data further comprises a command for a control subsystem to automatically switch the mechanical enhancement element to the anchoring state; wherein the mechanical enhancement element comprises an inflatable balloon configured to operate within at least a range of sub-anchoring pressures and a range of anchoring pressures, wherein to determine the action to be taken, the computing device is programmed by further executable instructions to determine, based at least partly on the procedure anomaly comprising a tissue-anomaly-related procedure anomaly, that the inflatable balloon is to be inflated to second anchoring pressure that is greater than a first anchoring pressure within the range of anchoring pressures, and wherein the feedback data comprises a command for a control subsystem to automatically increase the inflation pressure to the second anchoring pressure; wherein the tissue-anomaly-related procedure anomaly is based on detection of an at least partially-obstructed tissue anomaly; wherein the computing device is programmed by further executable instructions to obtain a second image using the visualization element, analyze the second image using the machine learning model to generate second classification output data, determine, based at least partly on the second classification output data, that the second image corresponds to no procedure anomaly, and cause presentation of a message regarding remediation of the procedure anomaly; wherein the mechanical enhancement element comprises an inflatable balloon, and wherein to determine the action to be taken, the computing device is programmed by further executable instructions to determine that a control subsystem is to change a degree of inflation of the inflatable balloon to at least one of a lower degree of sub-anchoring pressure, a higher degree of sub-anchoring pressure, an anchoring pressure, or a deflated state; wherein the computing device is programmed by further executable instructions to obtain a second image of a second portion of the mechanically-enhanced tissue from the internal imaging device, analyze the second image using a second machine learning model trained to detect tissue anomalies, determine, based at least partly on results of analyzing the second image using the second machine learning model, that the second image corresponds to a tissue anomaly, and perform a remedial action comprising at least one of generation of a command to a control subsystem to increase pressure of an inflatable balloon of the internal imaging device to an anchoring pressure, and display of a message instructing an operator to increase pressure of an inflatable balloon of the internal imaging device to an anchoring pressure; wherein the computing device is programmed by further executable instructions to obtain a second image of a second portion of the mechanically-enhanced tissue from the internal imaging device, analyze the second image using a second machine learning model trained to detect tissue anomalies, determine, based at least partly on results of analyzing the second image using the second machine learning model, that the second image corresponds to a tissue anomaly, and perform a remedial action comprising at least one of generation of a command to a control subsystem to switch the mechanical enhancement element to an anchoring state, and display of a message instructing an operator to switch a mechanical enhancement element of the internal imaging device to an anchoring state; wherein the computing device is programmed by further executable instructions to obtain a second image of a second portion of the mechanically-enhanced tissue from the internal imaging device, analyze the second image using a second machine learning model trained to detect tissue anomalies, determine, based at least partly on results of analyzing the second image using the second machine learning model, that the second image corresponds to a tissue anomaly, and perform a remedial action comprising at least one of generation of a command to a control subsystem to deflate an inflatable balloon of the internal imaging device, and display of a message instructing an operator to deflate an inflatable balloon of the internal imaging device; wherein to determine that the second image corresponds to a tissue anomaly, the computing device is programmed by further executable instructions to determine that the second image corresponds to at least one of a perforation of the portion of tissue, a major bleeding, and a serious adverse condition of the portion of tissue; and wherein the procedure anomaly comprises a tissue-anomaly-related procedure anomaly associated with a non-topographical feature of the portion of tissue.

### BRIEF DESCRIPTION OF THE DRAWINGS

Embodiments will now be described with reference to the following drawings, which are provided by way of example, and not limitation. Like reference numerals indicate identical or functionally similar elements.

FIG. 1 illustrates an internal imaging system with a mechanical enhancement element according to some embodiments.

FIG. 2 illustrates subsystems of the internal imaging system of FIG. 1 for analyzing images using a machine learning model and initiating remedial actions in response to detection of anomalies according to some embodiments.

FIG. 3 is a flow diagram of an illustrative routine for using a procedure analysis model to dynamically manage an internal imaging procedure according to some embodiments.

FIG. 4 illustrates use of an internal imaging system in a transverse colon in un-stretched and stretched states according to some embodiments.

FIG. 5 illustrates views of an intestinal lumen in an unaltered state and various mechanically-altered states according to some embodiments.

FIG. 6 illustrates views of an internal imaging system in an intestinal lumen being withdrawn at various speeds according to some embodiments.

FIG. 7 illustrates views of an internal imaging system in a transverse colon with automatic application of anchoring pressure according to some embodiments.

FIGS. 8A and 8B illustrate user interfaces presenting various alerts and images from an internal imaging system according to some embodiments.

FIGS. 9A, 9B, and 9C illustrate user interfaces presenting various alerts and images from an internal imaging system according to some embodiments.

FIG. 10 is a flow diagram of an illustrative process for training an artificial intelligence-based detection system using images generated using an imaging device with a mechanical enhancement element according to some embodiments.

FIG. 11 is a block diagram of illustrative data flows and interactions between imaging systems and an artificial intelligence training system according to some embodiments.

FIG. 12 is a block diagram of an illustrative machine learning model for analyzing tissue images according to some embodiments.

FIG. 13 is a block diagram of an illustrative computing system configured to implement training of machine learning models according to some embodiments.

### DETAILED DESCRIPTION OF ILLUSTRATIVE EMBODIMENTS

The present disclosure is directed to an artificial-intelligence-based system for analyzing and managing internal imaging procedures performed using internal imaging devices that mechanically alter tissue.

Some conventional internal imaging systems, such as endoscopes, generate imagery of internal body tissue. Illustratively, an endoscope may generate an image of a lumen of a patient's large or small intestine or other portions of the gastro-intestinal track. For example, video colonoscopes such as the CF-QH190L/I colonoscope, operating with Video System Center OLYMPUS CV-190, commercially available from Olympus Europe GmbH, of Amsinckstraße 63, 20097, Hamburg, Germany, or the EC38-i10L colonoscope, operating with endoscope system EPK-i5000, commercially available from Pentax Medical GmbH of Julius Vosseler st. 104, 22527, Hamburg, Germany, or the EC-760R-V/M colonoscope, operating with endoscope system ELUXEO 7000, commercially available from FUJIFILM Europe GmbH of Heesenstrasse 31 D-40549 Düsseldorf, Germany, are optical visualization devices capable of viewing and recording video images of the gastro-intestinal track.

Health care professionals may use the images to identify regions of interest on the tissue, including tissue abnormalities such as polyps or other lesions, inflammation, gastrointestinal bleeding, ulcers, etc. To aid the health care professionals in identifying regions of interest, various aids may be employed to mechanically enhance the tissue being imaged. Some internal imaging devices, such as colonoscopes or other endoscopes, may include a mechanical enhancement element, such as a balloon or other structure, that can exert pressure on the tissue to regulate or otherwise manipulate the tissue (e.g., cause stretching of the tissue when the structure is axially displaced relative to the lumen through a force applied to the endoscope). For example, internal imaging devices with mechanical enhancement elements such as the inflatable balloon of the G-EYE® endoscope commercially available from Smart Medical Systems Ltd. of 5 Hanofar St., Raanana, Israel, the EndoRings™ endoscope tip attachment commercially available from EndoAid, Ltd. of 43 Haeshel St., 3088900, Caesarea, Israel, or the EndoCuff® endoscope tip attachment commercially available from Olympus Europe GmbH, of Amsinckstraße 63, 20097, Hamburg, Germany, may be used in colonoscopy to mechanically enhance the intestinal lumen. For tissue with a folded or otherwise uneven topography, such as an intestinal lumen and in particular a colon lumen, such stretching and/or other tissue mechanical manipulation can flatten out, unfold and stretch the tissue and improve visibility of polyps, lesions, and other regions of interest. Images may be captured of tissue that has been mechanically enhanced by application of pressure from—and movement of—the balloon or other mechanical enhancement element. The images may be analyzed by health care professionals to identify tissue abnormalities or other regions of interest.

Use of internal imaging devices with mechanical enhancement elements may require a health care professional or other user or operator (collectively referred to herein as health care professional) to set and adjust various operational parameters of the mechanical enhancement element (e.g., degree of inflation) and implement various operational parameters of the internal imaging device itself (e.g., determine and implement the appropriate speed to advance and/or withdraw the internal imaging device). Management of these operational parameters may be required concurrently with review of live or substantially live images generated by the internal imaging device.

Aspects of the present disclosure relate to dynamic, artificial-intelligence-based analysis and management of internal imaging procedures, such as colonoscopies, using an internal imaging device with a mechanical enhancement element that alters tissue from its natural state or orientation such that regions of interest on the tissue may be more clearly distinguished from the surrounding tissue. Images of such internal imaging procedures are used to train an artificial intelligence system to detect anomalies during the procedures. Based on detection of such anomalies, various remedial actions may be taken with respect to the internal imaging device and/or mechanical enhancement component. Generally described, remedial actions taken with respect to the internal imaging device and/or mechanical enhancement component include actions that cause, or are intended to cause, changes to operational parameters and/or use of the internal imaging device and/or mechanical enhancement component. For example, such remedial actions may include providing feedback to the health care professional operating the internal imaging device to change an operational parameter or manner of use, and/or causing a control system to automatically adjust operational parameters of the internal imaging system to address the anomalies.

In some embodiments, images (including still images and/or videos) may be captured during internal imaging procedures performed using an internal imaging device with a mechanical enhancement element. Some images may be of tissue that has been mechanically enhanced by application of an optimal or otherwise desired degree of pressure from—and speed of movement of—the balloon or other mechanical enhancement element. Other images may be of tissue that has not been mechanically enhanced, or that has been mechanically enhanced to a greater or lesser degree than optimal or otherwise desired. Still other images may be of tissue responding to a greater or lesser speed of movement of the mechanical enhancement element than the optimal or otherwise desired speed of movement. Other images may be of tissue anomalies (e.g., polyps or other lesions, bleeding, etc.) where the internal imaging device is not stopped to evaluate the tissue anomalies. Further images may be of portions of procedures in which a visualization element of the internal imaging system has been maneuvered or otherwise deflected from a straight-ahead or default orientation at a greater rate and/or a greater number of times than optimal or otherwise desired. Data regarding operational parameters of the internal imaging device during capture of the respective images may be recorded, such as inflation metrics representing the degree of inflation of the balloon.

The images may be annotated or “labeled” into various classes, such as: too little stretching of tissue, too much stretching of tissue, too great speed of withdrawal, too slow speed of withdrawal, too much deflection of the visualization element, malfunction of the mechanical enhancement element (e.g., a hole in the balloon), unstopped withdrawal while passing through a particular abnormality (e.g., major bleeding, large polyp, etc.), acceptable amount of tissue stretching, acceptable speed of withdrawal, other classes, or some combination thereof. The example labels and classes described herein are illustrative only, and are not intended to be limiting, required, or exhaustive. In some embodiments, fewer, additional, and/or alternative labels and classes may be used. The labeled images may then be used to train a machine learning model for use in automatically detecting procedure anomalies.

With reference to an illustrative embodiment, a number of endoscope procedures may be performed using an endoscope with a balloon or other mechanical enhancement element. Some endoscope procedures or portions thereof may not include any anomalies (e.g., the amount of tissue alteration is optimal or otherwise within a desired range, the speed of endoscope withdrawal may be optimal or otherwise within a desired range, etc.), while other endoscope procedures or portions there may include one or more anomalies (e.g., the amount of tissue alteration such as stretching may be non-optimal or otherwise outside of a desired range, the speed of withdrawal may be non-optimal or otherwise outside of a desired range, the visualization element is deflected at greater than a desired rate, etc.). Annotated images from those procedures may be used as training data for an artificial intelligence system, such as a system that incorporates a machine learning model (e.g., a convolutional neural network or “CNN”). Parameters of the machine learning model may be initialized, and the machine learning model may be trained in an iterative manner by processing training data images and producing detection output. The detection output may be classification output indicating which images and/or portions of video are likely to show anomalies. The detection output may be evaluated against the annotations for the image to determine the degree to which the detection output differs from the desired output represented by the annotations. Based on this evaluation for one or more images, the parameters of the machine learning model may be modified. For example, a gradient descent algorithm may be used to determine the gradient of a loss function computed based on the training output and the desired output. The gradient may then be used to adjust the parameters of the machine learning model in particular directions and/or magnitudes so that subsequent processing of the same images will produce detection output closer to the desired output. This process may be repeated in an iterative manner until a desired stopping point is reached. For example, the desired stopping point may correspond to satisfaction of an accuracy metric, exhaustion of a quantity of training time or training iterations, etc.

In some embodiments, a number of endoscope procedures may be performed by a highly-skilled health care professional using an internal imaging device with a mechanical enhancement element. Images from such procedures may depict consistent achievement of optimal or otherwise desired tissue alteration, withdrawal speed, and the like. Images from such procedures may be used alone, or with images of procedure anomalies (e.g., intentional procedure anomalies caused for training purposes, and/or procedure anomalies otherwise captured during procedures) to train a machine learning model regarding the desired degree of tissue alternation, withdrawal speed, and the like. The model may be implemented as an anomaly detection model such that when presented with images that deviate from the desired degree of tissue alternation, withdrawal speed, etc., the model generates output indicating detection of an anomalous condition.

Additional aspects of the present disclosure relate to using a machine learning model, such as a model trained as described above and in greater detail elbow, in an artificial-intelligence-based system during internal imaging procedures performed using internal imaging devices configured with mechanical enhancement elements. Such an artificial-intelligence-based system may be referred to as a dynamic procedure management system, or as a subsystem of a larger internal imaging system. By using the dynamic procedure management system in clinical procedures with an internal imaging device having a mechanical enhancement element such as a balloon endoscope, the dynamic procedure management system can provide feedback to users or to a control system that automatically adjusts operational parameters of the internal imaging device. For example, if a particular procedure anomaly is detected, such as too little stretching of tissue, a remedial action can be triggered. In some embodiments, the remedial action may be presentation of an alert or other message. The alert may indicate that the tissue is not stretched to a desirable degree, and/or it may indicate a specific action to be taken by the health care professional to address the anomaly (e.g., increase the degree of inflation of the balloon, check whether the balloon is inflated or has a hole). In some embodiments, the remedial action may be alteration of an operational parameter of the internal imaging system, such as an instruction to a control subsystem to address the anomaly (e.g., increase the degree of inflation of the balloon).

Further aspects of the present disclosure relate to using a machine learning model or other method of detecting tissue anomalies in conjunction with a machine learning model configured to detect procedure anomalies. For example, a machine learning model may be trained using images of mechanically-enhanced or otherwise mechanically-altered tissue to learn features that distinguish tissue anomalies (e.g., polyps or other lesions, perforations, etc.) from other tissue regions. The tissue anomaly model may be used during internal imaging procedures to detect tissue anomalies. If a particular tissue anomaly (e.g., large polyp, tumor, major bleeding, perforation) is detected and the internal imaging device does not stop to inspect the tissue anomaly (e.g., the internal imaging device continues to be withdrawn), a procedure anomaly may be triggered and a remedial action may be performed. In some embodiments, the remedial action may include presentation of an alert to inspect the potential tissue anomaly. In some embodiments the remedial action may include automatic adjustment of an operational parameter of the imaging device, such as inflation of a balloon to anchoring pressure in order to stop withdrawal of the imaging device and permit evaluation of the tissue anomaly, or deflation of the balloon. In some embodiments, a single machine learning model may be trained to detect both procedure anomalies and tissue anomalies.

Various aspects of the disclosure will now be described with regard to certain examples and embodiments, which are intended to illustrate but not limit the disclosure. Systems, methods, and components can be used in different embodiments. Some embodiments are illustrated in the accompanying figures; however, the figures are provided for convenience of illustration only, and should not be interpreted to limit the inventions to the particular combinations of features shown. Rather, any feature, structure, material, step, or component of any embodiment described and/or illustrated in this specification can be used by itself, or with or instead of any other feature, structure, material, step, or component of any other embodiment described and/or illustrated in this specification. Nothing in this specification is essential or indispensable.

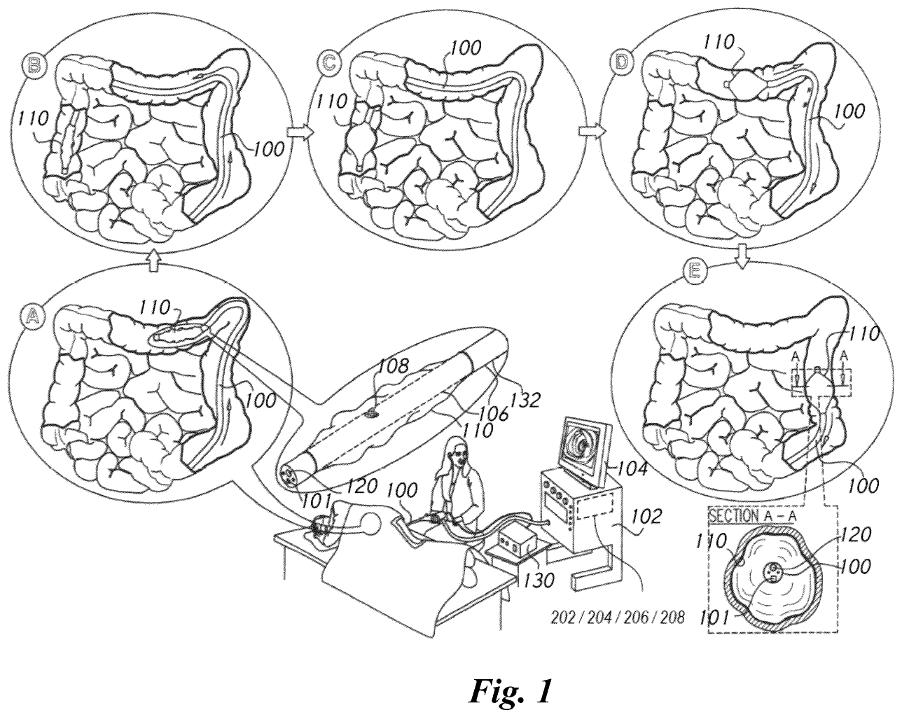

Reference is now made to FIG. 1, which is a simplified illustration of an internal imaging system in which aspects of the present disclosure may be implemented. In the illustrated example, the internal imaging system is an endoscope system. The terms “endoscope” and “endoscopy” are used throughout and refer to apparatus and methods which operate within body cavities, passageways and the like, such as, for example, the small intestine and the large intestine. The term “forward” refers to the remote end of an endoscope, accessory or tool furthest from the operator or to a direction facing such remote end. The term “rearward” refers to the end portion of an endoscope, accessory or tool closest to the operator, typically outside an organ or body portion of interest or to a direction facing such end portion. The term “pressure” generally refers to measurements indicated in millibars above ambient (atmospheric) pressure.

FIG. 1 illustrates the general structure and operation of an embodiment of an endoscope offering mechanical enhancement (e.g., stretching) of tissue and providing images of the tissue via a visualization element (e.g., a camera and illumination source). In some embodiments, as shown, such an endoscope may be a balloon endoscope.

The endoscope 100 shown in FIG. 1 has a visualization element, implemented as a charge-coupled device (“CCD”) 101, at a forward end of the endoscope 100. The CCD 101 is connected to an internal imaging system 102, such as an endoscope system, that may include a monitor 104. Alternatively, CCD 101 may be replaced by any other suitable visualization element. The endoscope system 102 may be configured with artificial-intelligence-based image analysis functionality, such as a dynamic procedure management subsystem 204 that includes a machine learning model trained to detect procedure anomalies in images of mechanically-enhanced tissue from an imaging subsystem 202. Based on results of analyzing the images and detecting procedure anomalies, the dynamic procedure management subsystem 204 may cause a user interface subsystem 206 to present alerts or other notifications, and/or the dynamic procedure management subsystem 204 may cause a control subsystem 208 to automatically adjust an operational parameter of the endoscope 100.

In some embodiments, the endoscope 100 may be an EC38-i10L video colonoscope, a G-EYE38-i10L balloon-colonoscope, an EG34-i10 gastroscope or a VSB-2990i video enteroscope, the endoscope system 102 may be a console including one or more computing devices, such as an EPK-i5010 video processor, and the monitor 104 may be a SONY LMD-2140MD medical grade flat panel LCD monitor, all commercially available from Pentax Europe GmbH, 104 Julius Vosseler St., 22527 Hamburg, Germany.

In some embodiments, as described in Published PCT Application WO 2011/111040, published on Sep. 15, 2011, the disclosure of which is hereby incorporated by reference, the endoscope 100 has an outer sheath 106 which may be provided with at least one balloon inflation/deflation aperture 108. The aperture 108 may communicate with the interior of an inflatable/deflatable balloon 110, sealably mounted on outer sheath 106, and with an interior volume of the endoscope 100, which in some endoscopes may be sealed from the exterior other than via a leak test port at a rearward portion of the endoscope. In some embodiments, the interior volume generally fills the interior of the endoscope 100 which is not occupied by conduits and other elements extending therethrough.

It is appreciated that a gas communication path may extend between the rearward portion of the endoscope to a balloon volume at the interior of inflatable/deflatable balloon 110. It is a particular feature of this embodiment that the interior volume provides a gas reservoir, enabling quick pressurization and depressurization of balloon 110 and a directly coupled pressure buffer operative to reduce the amplitude of pressure changes inside the balloon 110 resulting from corresponding changes in balloon volume. It is appreciated that having a gas reservoir, such as the interior volume, in inflation propinquity to balloon 110 as described herein, also provides inflation pressure buffering for balloon 110 and enables enhanced stability and accuracy to be achieved in the pressurization of the inflated balloon volume. In some embodiments, inflatable balloon 110 is directly coupled to a gas reservoir having a volume typically 3-7 times higher than the inflated balloon volume.

Alternatively, the interior of balloon 110 may communicate with a fluid flow passageway other than interior volume of the endoscope 100, such as, for example, a fluid conduit or other conduit, such as a conventional dedicated balloon inflation/deflation channel and aperture 108 may be obviated.

Inflatable/deflatable balloon 110 may be inflated and/or deflated via the interior volume of the balloon endoscope 100 by a balloon inflation/deflation system 130, which constitutes a balloon inflation and/or deflation subsystem of the endoscopy system of FIG. 1. An example of such a balloon inflation/deflation system 130 is the NaviAid SPARKC Inflation System product, available from Smart Medical System Ltd. of 5 Hanofar street, Raanana, Israel.

As shown, balloon 110 may be sealably mounted over a forward portion of endoscope 100, overlying outer sheath 106. In some embodiments, outer sheath 106 includes a tubular scaling sheath 132, overlying a reinforcement mesh, which serves to maintain the interior volume of endoscope 100 against collapse during bending thereof. Instrument channel 120 and may extend inwardly through the interior volume of endoscope 100. Other conduits and other elements may also extend through this interior volume. It is further appreciated that notwithstanding the fact that various conduits may extend through the interior volume 106, their presence does not result in fluid communication between the interior volume and the interior of any conduit extending therethrough.

Forwardly of tubular scaling sheath 132, outer sheath 106 may be formed of a tubular sealing bending rubber sheath, which also seals the interior volume from the exterior of endoscope 100. Illustratively, the bending rubber sheath may be a silicone bending rubber sheath part number SPRBSS11, PVC bending rubber sheath part number SPRBSP11, or a Viton bending rubber sheath part number SPRBSV11, all commercially available from Endoscope Repairs Inc. of 18205 North 51st Avenue, Suite 107, Glendale AZ, 85308 USA. Aperture 108 may be formed in the sheath. It is appreciated that plural apertures 108 may be provided for gas communication between the interior of inflatable/deflatable balloon 110 and the interior volume of endoscope 100.

The bending rubber sheath may overlay a selectably bendable reinforcement mesh, which is selectably bendable in response to operator manipulation of steering knobs (not shown) at a rearward portion of endoscope 100, and protects the forward selectably bendable portion of endoscope 100 against collapse during bending thereof. Instrument channel 120 and/or other elements extend interiorly of the selectably bendable reinforcement mesh, through the interior volume of the endoscope.

It is appreciated that a gas communication path may extend through the interior volume and aperture 108 to balloon volume at the interior of inflatable/deflatable balloon 110.

Advantageously, the illustrated arrangement provides secure and stable mounting of balloon 110 onto existing rigid mounting elements of the endoscope without the requirement of additional rigid mounting elements which could limit the flexibility of the endoscope. The resulting structure described above is both suitable for conventional reprocessing and provides a balloon-equipped endoscope which does not normally require balloon replacement.

In some embodiments, inflatable/deflatable balloon 110 is inflated and/or deflated via the interior volume of the balloon endoscope 100. The available cross section of the interior volume for inflation/deflation of the balloon 110 may be 15-50 square millimeters, which in some embodiments may be approximately 6-30 times greater than the cross section of balloon inflation channels employed in the prior art. The interior volume of the endoscope 100 may thus function as a gas reservoir directly coupled to the balloon, enabling inflation and deflation of the balloon 110 to take place, as well as fast and dynamic change in balloon pressure.

In some embodiments, the configuration of inflatable/deflatable balloon 110 is generally characterized as follows: balloon 110 is formed of a biocompatible polymer of thickness in the range of 10-75 micron, and potentially in the range of 20-50 micron. In some embodiments, the stretchability of the balloon 110 may be described as a non-linear function of the balloon internal pressure.

The balloon 110 may be relatively un-stretchable under low operative internal pressures and relatively stretchable under high operative internal pressures. For example, the balloon is not stretchable beyond 3% under relatively low internal pressures up to approximately 10 millibar and is stretchable beyond 6%-20% under relatively high internal pressures of approximately 60-80 millibar, respectively. An example of a balloon providing the aforementioned non-linear stretchability as function of balloon internal pressure is a balloon formed by blow-molding, having length of 110 millimeter and diameter of 48 millimeter when inflated to a pressure of 10 millibar, and having wall thickness of 25-35 micron. Preferable materials of balloon 110 include biocompatible polymer formulae, nylon or silicon.

The thickness and dimensions of balloon 110 may be configured to minimize interference with endoscope performance parameters when balloon 110 is deflated, such as bendability and ease of advancement, while providing long-term usability of the balloon-equipped endoscope during repeated endoscopy procedures and conventional reprocessing cycles, without requiring replacement of balloon 110.

Balloon 110 may have an overall length of 50-130 mm. The rearward and forward ends of balloon 110 may be generally cylindrical and have a fixed inner cross-sectional radius R1, when forming part of balloon endoscope 100. In some embodiments, RI is preferably between 4 and 7 mm so as to tightly engage the adjacent portions of the endoscope.

In operation, the endoscope 100 may be inserted, with balloon 110 in a deflated state, into a body passageway, such as a patient's large intestine. Stage A in FIG. 1 shows the endoscope 100 located in the transverse colon of the patient with balloon 110 in a deflated state and stage B shows the endoscope advanced through the patient's colon, to a location just rearwardly of the cecum with balloon 110 in a deflated state. Endoscopic inspection of the interior of the colon may take place during insertion of the endoscope.

In stage C, while the endoscope is not yet moved from its position in stage B, the balloon 110 may be inflated to an intermediate pressure state. Such an intermediate pressure state may be a sub-anchoring, slidable frictional engagement pressure which is sufficient to provide frictional engagement between the outer surface of the balloon 110 and the inner surface of the colon engaged thereby but less than a pressure which anchors the balloon 110 thereat. In some embodiments, balloon 110 is inflatable by balloon inflation/deflation system 130 to various pressures including an anchoring pressure and multiple selectable intermediate pressures.

Thereafter, the operator pulls the endoscope 100 rearwardly, while the balloon 110 is at the aforesaid sub-anchoring slidable frictional engagement pressure, thereby stretching the colon axially along its length and at least partially unfolding natural folds of the colon. Visual inspection of the colon may take place during the aforesaid retraction of the endoscope while the colon adjacent the forward end of the endoscope is axially stretched forwardly thereof. The aforesaid methodology of retracting the endoscope and thus stretching the colon and visually inspecting the interior of the colon while it is stretched can be carried out repeatedly along the colon from the cecum all of the way to the anus, such that the entire colon is systematically visually examined while each portion being examined is in a stretched state.

This inspection is shown generally in FIG. 1 at stage C when the forward end of endoscope 100 is located in the ascending (right) colon, thereafter at stage D when the forward end of endoscope 100 is located in the transverse colon and thereafter at stage E when the forward end of endoscope 100 is located in the descending (left) colon. Visual inspection of the colon while systematically axially stretching it to at least partially open, flatten and/or unfold the folds, enables detection of polyps and other potential and actual pathologies which might otherwise go undetected.

Stretching of tissue can also help to enhance visual aspects of non-topographical features. Such non-topographical enhancement can help non-topographical features become more pronounced, or become visible for the first time. This effect can provide additional opportunities for visual inspection that would not be practical or possible in images of non-mechanically-enhanced tissue. For example, visual aspects such as color, texture, transparency, contour and the like may be altered or visualized more readily in images of mechanically-enhanced tissue. Such non-topographical enhancement can aid in visualization of flat or depressed polyps. In some embodiments, subsurface features such as blood vessels become clearly distinguishable after application of mechanical enhancement.

For the purposes of the present disclosure, visual inspection is inspection in which a clear line of sight is required or desirable, for example inspection in the IR or visible band, as distinguished from inspection in which a clear line of sight is not relevant, such as some types of X-ray inspection.

Balloon 110 may be configured for generally circumferentially uniform slidable frictional engagement with the interior wall of a body passageway, typically a tubular body portion, such as the colon, when inflated to a generally circumferentially uniform slidable frictional engagement pressure and displaced axially along said body passageway. This circumferentially uniform slidable frictional engagement is shown, for example in section A-A in FIG. 1.

Rearward axial displacement of balloon 110 in a body passageway under inspection when the balloon is in slidable frictional engagement with the interior wall of the body passageway, and when being in generally circumferentially uniform slidable frictional engagement with the interior wall of the body passageway, provides at least partial removal of materials and fluids in the body passageway from the interior wall just prior to visual inspection thereof. Such materials and fluids may include, for example, food, feces, body fluids, blood and irrigation liquids injected by the endoscope 100 and could, if not removed, interfere with the visual inspection.

The material and thickness of balloon 110 may be selected and configured such that balloon 110 is radially compliant and conformable to the inner circumferential contour of the body passageway at the balloon engagement location, as to allow generally circumferentially uniform slidable frictional engagement of balloon 110 with the body passageway under inspection. An example of such a radially compliant and conformable balloon is a balloon having wall thickness of 20-50 microns.

In some embodiments, the generally circumferentially uniform slidable frictional engagement pressure is in the range of 5-50 millibar, in a narrower range of 20-50 millibar, or in a still narrower range of 35-45 millibar.

Axial displacement of the endoscope balloon in generally circumferentially uniform slidable frictional engagement with the interior of the colon in order to achieve desired axial stretching of the colon may be in a range of 10-100 millimeters, in a narrower range of 15-70 millimeters, and sometimes in a narrower range of 30-60 millimeters.

In some embodiments, the axial stretching produced in the colon forwardly of CCD 101 of endoscope 100 may be at least 25%, at least 35%, at least 60%, or at least 100%.

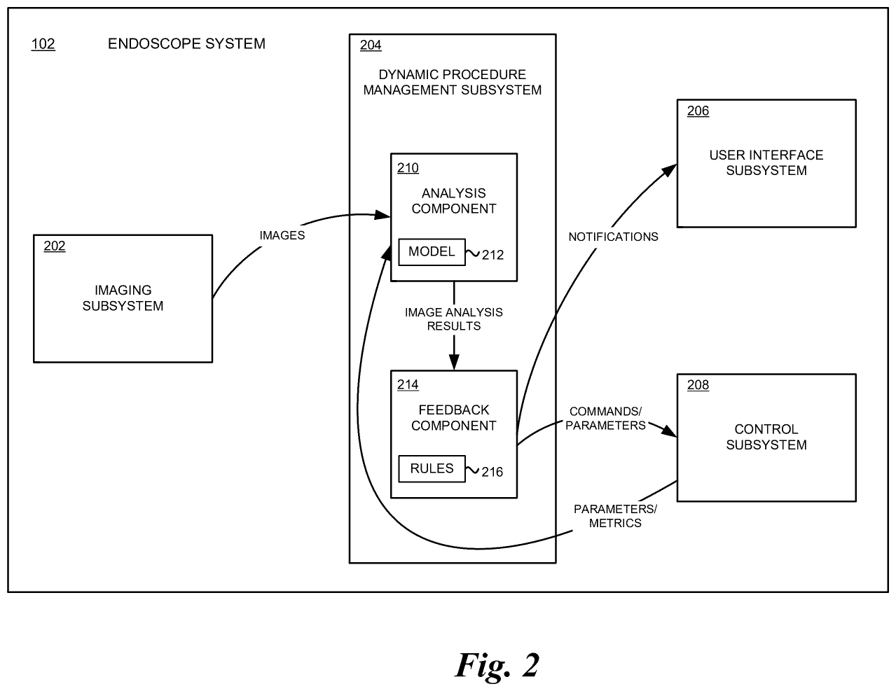

With reference now to FIGS. 2-7, 8A, 8B, 9A, 9B, and 9C (collectively “FIGS. 2-9C”), several examples and illustrations of the operation of the dynamic procedure management subsystem 204 and other subsystems of the endoscope system 102 will be described.

FIG. 2 is a simplified illustration of an endoscope system 102 with an imaging subsystem 202 to process images generated by the visualization element of the endoscope 100, a dynamic procedure management subsystem 204 to analyze the images and other data to detect anomalies, a user interface subsystem 206 to present images and alters regarding actions to be taken, and a control subsystem 208 to control operational parameters of the endoscope system and implement the adjustment based on detected anomalies. The imaging subsystem 202, dynamic procedure management subsystem 204, user interface subsystem 206, and control subsystem 208 may each be implemented using hardware, or a combination of hardware and software. For example, the endoscope system 102 may include one or more computer processors and memory within a housing, and the imaging subsystem 202, dynamic procedure management subsystem 204, user interface subsystem 206, and control subsystem 208 may be implemented by execution by the processor(s) of instructions stored in the memory.

In some embodiments, one or more of the imaging subsystem 202, dynamic procedure management subsystem 204, user interface subsystem 206, and/or control subsystem 208 may be implemented separately from other subsystems. For example, the endoscope system may include two or more physically separate devices, and each separate device may include a housing in which one or more processors and computer-readable memory may be included. One device may implement one or more of the subsystems (e.g., imaging subsystem 202, dynamic procedure management subsystem 204, user interface subsystem 206, and/or control subsystem 208), a second device may implement one or more of the remaining subsystems, and so on.

The imaging subsystem 202 may receive image data from the visualization element of the endoscope 100. For example, the endoscope 100 may provide streaming video and/or images captured during a procedure. The imaging subsystem 202 may perform various storage, processing, and/or routing tasks with the image data, such as by preparing the image data for presentation via the user interface subsystem 206. As shown in FIG. 2, the imaging subsystem 202 may provide image data to the dynamic procedure management subsystem 204 for analysis and detection of anomalies, implementation of remedial actions, and the like.

The dynamic procedure management subsystem 204 may include one or more components to provide various features described herein. In some embodiments, as shown, the dynamic procedure management subsystem 204 may include an analysis component 210 that uses a machine learning model 212 to analyze image data received from the imaging subsystem 202 and, optionally, additional data such as current operational parameters/metrics of the control subsystem 208 when the images were captured.

Results of the analysis may be provided to a feedback component 214 that determines which remedial action(s), if any, to initiate based on the results of the analysis. In some embodiments, the feedback component 214 may use a set of rules 216 that associate classification determinations made by the analysis component 210. For example, a classification of too little tissue stretching may be associated with a message that is sent to the user interface subsystem 206 to alert a health care professional to increase the degree of pressure in the balloon, and/or to increase the withdrawal speed of the endoscope 100. As another example, a classification of too much tissue stretching may be associated with a command to the control subsystem 208 to reduce the degree of pressure in the balloon. Examples of image analysis results and corresponding remedial actions are described in greater detail below.

The user interface subsystem 206 may be configured to control what is presented on the monitor 104. For example, the user interface system 206 may cause presentation of image data from the endoscope 100, alerts and/or user instructions from the dynamic procedure management subsystem 204, data regarding parameters of the control subsystem 208, etc.

The control subsystem 208 may be configured to manage operational parameters of the endoscope 100, balloon 110, balloon inflation/deflation system 130, etc. For example, the control subsystem 208 may be configured to receive commands from the dynamic procedure management subsystem 204 to increase/decrease inflation of the balloon 110. As another example, the control subsystem 208 may receive commands from an operator to increase/decrease inflation of the balloon 110, adjust the orientation of the visualization element of the endoscope, etc.

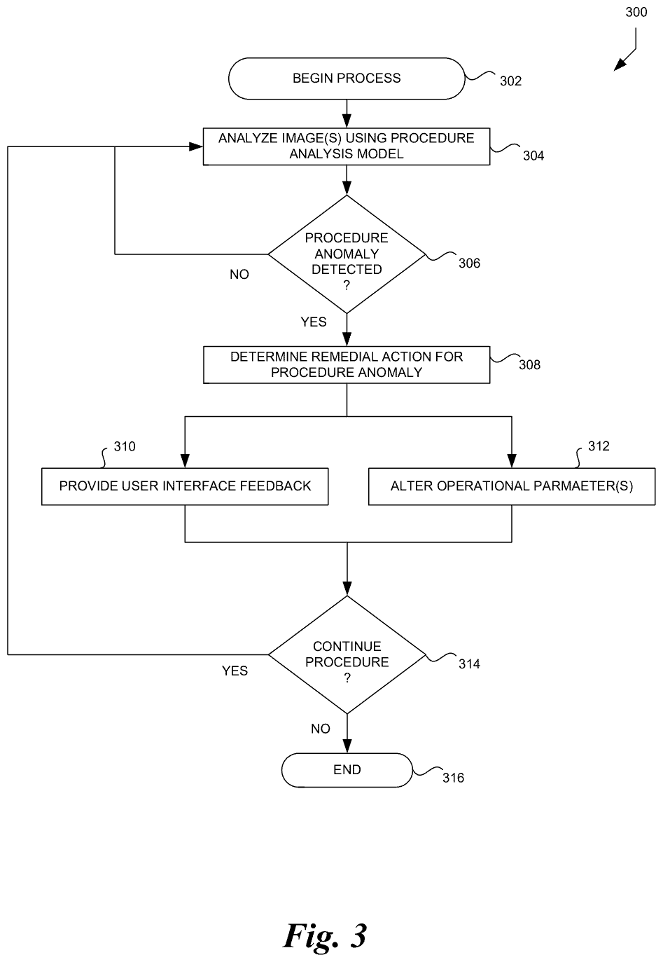

FIG. 3 is a flow diagram of an illustrative routine 300 that may be executed to manage an internal imaging procedure performed using an internal imaging device with a mechanical enhancement element. Advantageously, the routine 300 uses a machine learning model, for example machine learning model 212 of FIG. 2, to detect anomalies during the internal imaging procedure and implement remedial actions that can alert a health care professional during the procedure and/or automatically adjust operational parameters of the internal imaging routine to address the detected anomalies.

Although the routine 300 will be described with reference to analyzing individual images using a machine learning model, it will be appreciated that the routine may also or alternatively be performed using video. For example, individual frames of video may be handled substantially as described with respect to images.

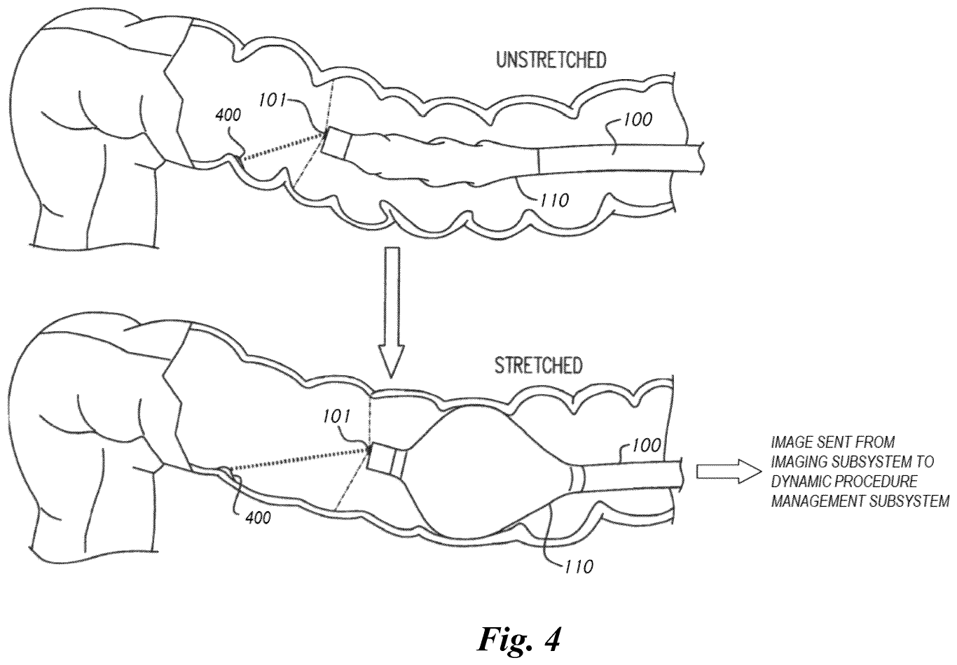

Portions of the routine 300 will be described with further reference to the illustrative procedure conditions, anomalies, and remedial actions shown in FIGS. 4-8A, 8B, 9A, 9B, and 9C (collectively “FIGS. 4-9C”).

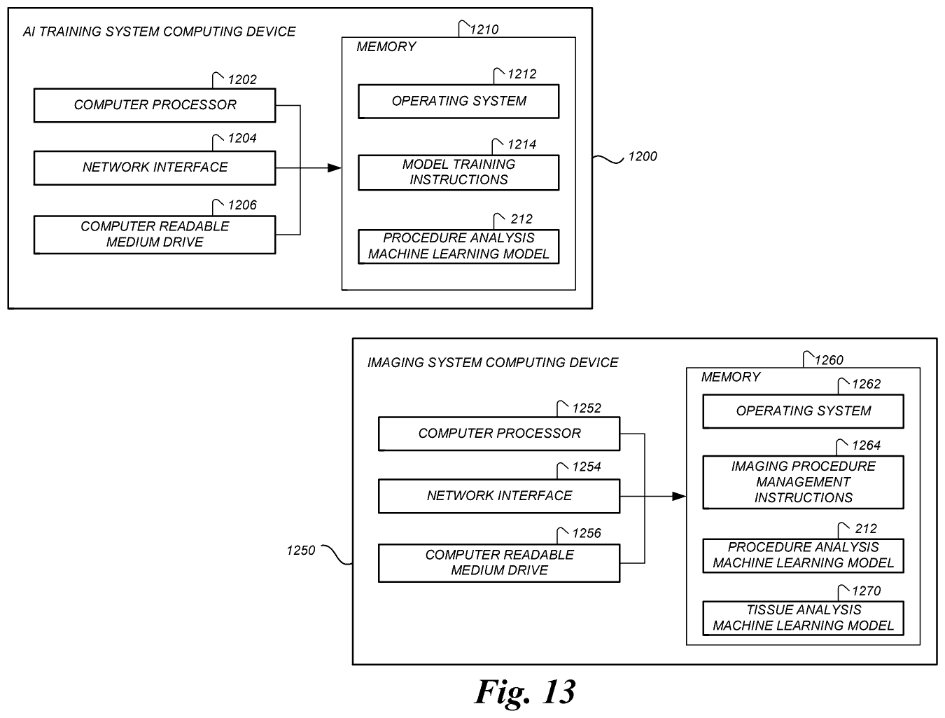

The routine 300 begins at block 302. The routine 302 may begin in response to an event, such as when the internal imaging system 102 begins operation, or in response to some other event. When the routine 300 is initiated, a set of executable program instructions stored on one or more non-transitory computer-readable media (e.g., hard drive, flash memory, removable media, etc.) may be loaded into memory (e.g., random access memory or “RAM”) of a computing device of the internal imaging system, such as the imaging system computing device 1250 shown in FIG. 13 and described in greater detail below. In some embodiments, the routine 300 or portions thereof may be implemented on multiple processors, serially or in parallel.

At block 304, the analysis component 210 or some other module or component of the dynamic procedure management subsystem 204 may analyze image data received from the imaging subsystem 202. The image data may originate from, or be derived from images generated by, the visualization element of the endoscope 100.

FIG. 4 illustrates an example of endoscope 100 being used to capture images of an inner lumen of a colon. As shown, the endoscope 100 may be advanced into the colon, and then the balloon 110 may be at least partially inflated (e.g., to sub-anchoring pressure). The endoscope 100 may then be retracted, which causes a flattening of the topography of the inner lumen of the colon. CCD 101 may capture images of the flattened topography, including region of interest such as a tissue anomaly 400. The image data generated by the CCD 101 may be received by the imaging subsystem 202, where in some embodiments pre-processing may be performed (photographic adjustments, scaling, compression, conversion to greyscale, etc.) prior to being provided to the analysis component 210.

Analysis of the image data may be performed using the machine learning model 212, which in the example of illustrative routine 300 may be a procedure anomaly detection model. The procedure anomaly detection model 212 may be trained to detect various anomalies during internal imaging procedures performed using mechanical enhancement elements, such as balloon endoscopes. As described above, use of a mechanical enhancement element may cause tissue to be advantageously altered (e.g., stretched, unfolded, flattened, etc.), thereby allowing for improved detection of tissue anomalies such as polyps, lesions, abnormal blood vessel formations, areas of bleeding, perforations, and the like. However, in some cases the mechanical enhancement may fall outside the range of what is optimal or desired.

For example, the internal imaging procedure may be performed using a tissue anomaly detection model that is trained on images of mechanically-enhanced tissue to detect tissue anomalies in tissue that has been mechanically enhanced to an optimal or otherwise desired degree. One example of the training and use of such a model is described in PCT Patent Application PCT/US21/23139, the disclosure of which is hereby incorporated by reference and made part of this specification. If the tissue in the current internal imaging procedure is not mechanically enhanced to the optimal or otherwise desired degree, the results of analyzing images of the tissue using such a tissue anomaly detection model may not be acceptable.

In some embodiments when a balloon endoscope is used, non-optimal or otherwise non-desirable mechanical enhancement of the tissue can have a number of different causes, such as too little inflation of the balloon, too great inflation of the balloon, too slow withdrawal of the endoscope, too fast withdrawal of the endoscope, too many adjustments to the direction of the visual element, and/or some combination thereof.

To detect and address non-optimal degrees of mechanical tissue enhancement and/or other procedure anomalies, the procedure anomaly detection model 212 may be trained to detect procedure anomalies based at least partly on images captured during the procedures. In some embodiments, additional data associated with the procedure may be used to detect procedure anomalies, such as data regarding operational parameters of the internal imaging device (e.g., inflation metrics). The procedure anomaly detection model 212 may generate output indicating a classification of input regarding a portion of the current procedure as being associated with a procedure anomaly. For example, procedure anomaly detection model 212 may generate output indicating whether input regarding a portion of the current procedure is indicative of too much stretching of the tissue, too little stretching of the tissue, speed of withdrawal is too great, speed of withdrawal is too slow, the visualization element is being adjusted too often (e.g., by angulation of the distal bending section of the endoscope too often and/or to a too large angulation angle), etc. The exemplary procedure anomalies detected using the procedure anomaly detection model 212 described herein are illustrative only, and are not intended to be limiting, required, or exhaustive. In some embodiments, additional, fewer, and/or alternative procedure anomalies may be detected.

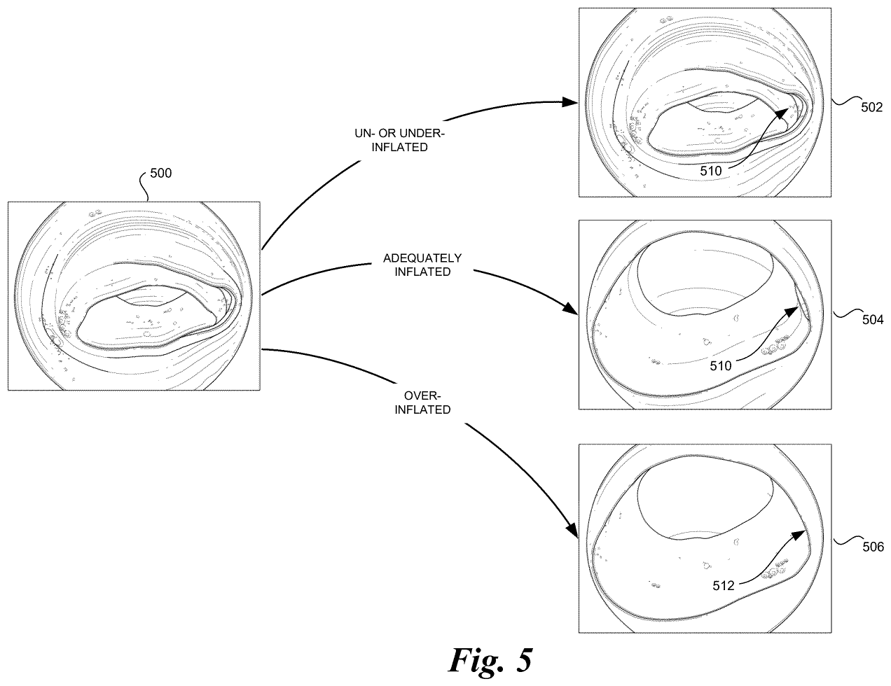

FIG. 5 illustrates examples of various degrees of tissue stretching, some of which may be indicative of a procedure anomaly and some of which may not. Image 500 shows a section of a colon without any mechanical enhancement of the tissue (e.g., without a balloon being inflated during endoscope withdrawal). During an internal imaging procedure, the tissue may be mechanically altered (e.g., by inflating the balloon to sub-anchoring pressure and withdrawing the endoscope with the balloon maintained in this sub-anchoring inflation state).

Image 504 shows the same section of the colon as image 500, but with a desired amount of mechanical enhancement applied. For example, a balloon may be inflated to a desired degree and/or the balloon endoscope may be withdrawn at a desired speed to stretch the tissue and unfold or otherwise flatten, stretch and/or smoothen the topography of the tissue. As shown in image 504, a tissue anomaly 510 becomes readily apparent with the desired degree of mechanical enhancement.

Image 502 shows the same section of the colon as images 500 and 504, but with less than a desired amount of mechanical enhancement applied. For example, a balloon may be inflated to less than a desired degree, or not inflated at all. As shown in image 502, the topography of the tissue is not as flat as the topography of the same section of the colon depicted in image 504. Because of this, the tissue anomaly 510 is not as apparent and/or differentiated from the surrounding tissue as in image 504.

Image 506 shows the same section of the colon as images 500, 502, and 504, but with greater than a desired amount of mechanical enhancement applied. For example, a balloon may be inflated to greater than a desired degree, and/or the balloon endoscope may be withdrawn at a faster speed than desired to stretch the tissue and unfold or otherwise flatten, stretch and/or smoothen the topography of the tissue. As shown in image 506, the topography of the tissue is significantly flatter than the topography shown in images 500 and 502, and even flatter than the topography in image 504. The topography has been flattened to such a degree that there is no longer an anomaly apparent in the image at area 512, corresponding to the portion of tissue where anomaly 510 is visible in the other images.

The procedure anomaly detection model 212 may be trained to analyze images such as those shown in FIG. 5, and generate output that classifies the images as likely depicting particular procedure anomalies or no anomaly. For example, when presented with image 502, the procedure anomaly detection model 212 may generate output indicating presence of an under-inflation or un-inflation anomaly. When presented with image 504, the procedure anomaly detection model 212 may generate output indicating no procedure anomaly being present. When presented with image 506, the procedure anomaly detection model 212 may generate output indicating presence of an over-inflation anomaly.

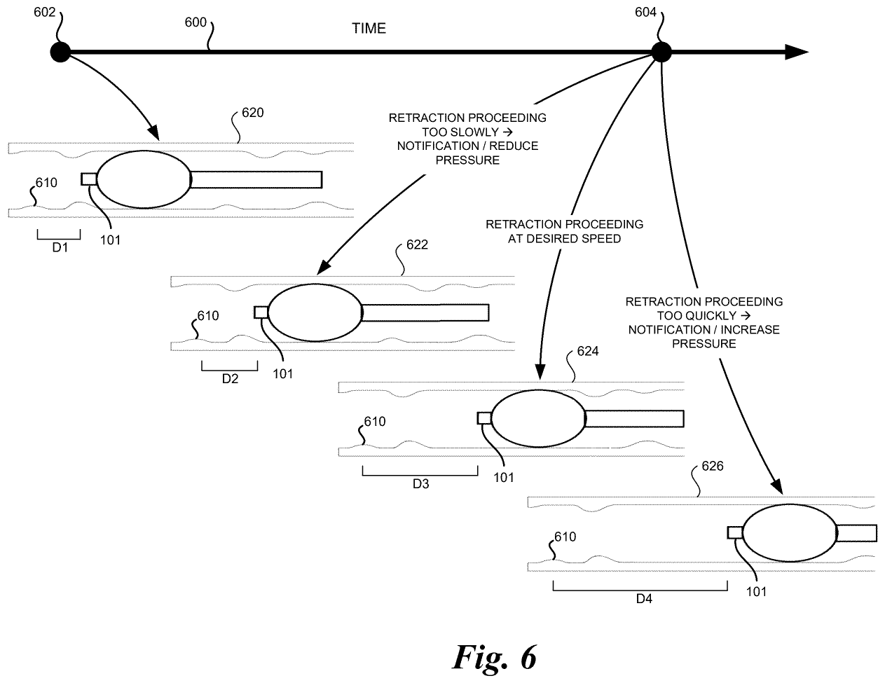

FIG. 6 illustrates examples of various degrees of withdrawal speed, also referred to as retraction speed, of the internal imaging device, some of which may be indicative of a procedure anomaly and some of which may not. Procedure timeline 600 includes time point 602 and subsequent time point 604. The procedure anomaly detection model 212 may be configured to detect withdrawal speed anomalies based on the images captured at the time points 602, 604.

Example 620 shows the CCD 101 of the internal imaging device positioned distance DI from topographical feature 610 at time point 602.

Example 622 shows the CCD 101 of the internal imaging device positioned distance D2 from topographical feature 610 at time point 604. Although D2>D1, which indicates that the internal imaging device has been retracted in the time period between time points 602 and 604, the retraction distance may be less than desired, which indicates that retraction is proceeding too slowly.

Example 626 shows the CCD 101 of the internal imaging device positioned distance D4 from topographical feature 610 at time point 604. Although D4>D2, which indicates that the internal imaging device has been retracted in the time period between time points 602 and 604 at a greater speed than in example 622, the retraction distance may be greater than desired, which indicates that retraction is proceeding too quickly.

Example 624 shows the CCD 101 of the internal imaging device positioned distance D3 from topographical feature 610 at time point 604, where D4>D3>D2>D1. In this example, distance D3 may be a desired distance and may indicate that the internal imaging device has been retracted at a desired retraction speed.

The procedure anomaly detection model 212 may be trained to analyze images at different time points, such as time points 602 and 604 as shown in FIG. 6, and generate output that classifies the images as likely depicting particular anomalies or no anomaly. For example, when presented with an image captured at distance D2 at time point 604, the procedure anomaly detection model 212 may generate output indicating presence of a slow retraction anomaly, also referred to as a low withdrawal speed anomaly. When presented with an image captured at distance D3 at time point 604, the procedure anomaly detection model 212 may generate output indicating no procedure anomaly being present. When presented with an image captured at distance D4 at time point 604, the procedure anomaly detection model 212 may generate output indicating presence of a fast retraction anomaly, also referred to as a high withdrawal speed anomaly.

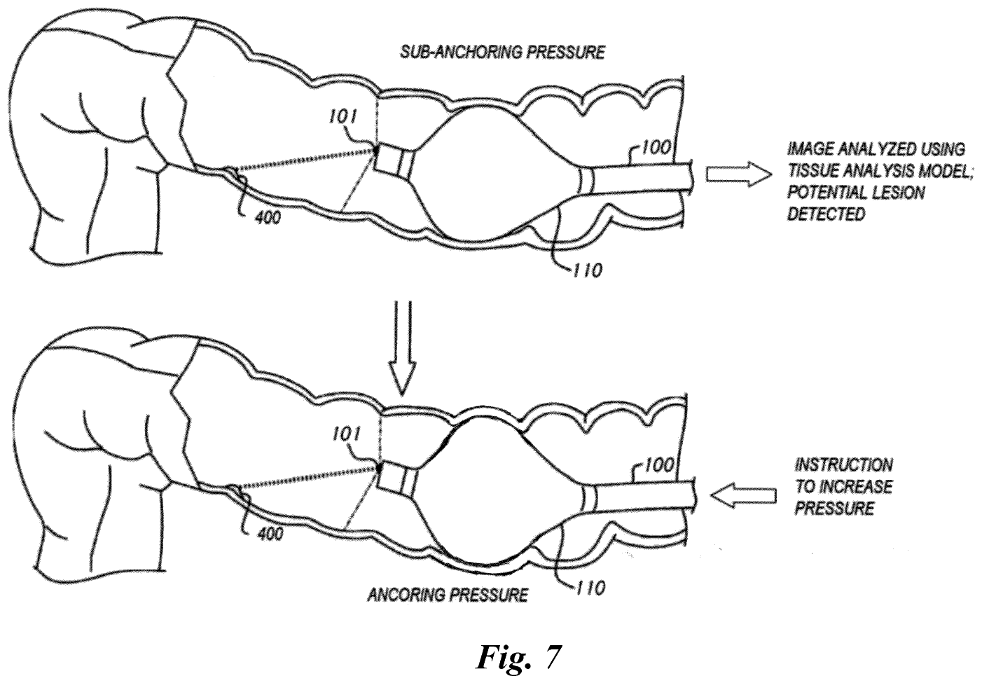

FIG. 7 illustrates detection of a tissue anomaly, which may trigger a remedial action alone, or in combination with detection of a procedure anomaly. As shown, an endoscope 100 has a balloon 110 inflated to a sub-anchoring pressure. Images captured by the CCD 101 may be analyzed using a tissue anomaly detection model, also referred to as a tissue analysis model, and may result in detection of tissue anomaly 400. Procedure anomaly detection model 212 may generate output indicating that endoscope 100 continues to be retracted. This combination may trigger detection of a procedure anomaly because a tissue anomaly 400 has been detected but retraction of the endoscope 100 has not been stopped to evaluate the tissue anomaly 400. Such a procedure anomaly is referred to also as a tissue-anomaly-related procedure anomaly.

Returning to FIG. 3, at decision block 306 the analysis component 210 or some other module or component of the dynamic procedure management subsystem 204 may determine whether a procedure anomaly has been detected based on results of analyzing images and optionally other data using the procedure anomaly detection model 212. For example, based on the analyses described above, the analysis component 210 may determine whether one of the described anomalies has been detected. If so, the routine 300 can proceed to block 308. Otherwise, the routine 300 may return to block 304 for continued analysis of the internal imaging procedure.

At block 308, the feedback component 214 or some other module or component of the dynamic procedure management subsystem 204 may determine a remedial action to be performed in response to the procedure anomaly. In some embodiments, the feedback component may include a set of rules 216 that associated remedial actions with procedure anomalies. Detection of different procedure anomalies may trigger different remedial actions. In addition, the remedial actions may be implemented in different modalities, such as visual alerts and/or instructions to the operator, audible alerts and/or instructions to the operator, automatic modification of operational parameters, or some combination thereof. If the remedial action is presentation to a health care professional or if the health care professional is to be presented with a message regarding the remedial action to be taken, the routine 300 can proceed to block 310. If the remedial action additionally or alternatively includes alteration of one or more operational parameters, the routine 300 can proceed additionally or alternatively to block 312.

FIG. 5 shows example procedure anomalies in which too much mechanical alteration is applied to imaged tissue (e.g., in image 506) and in which not enough mechanical alteration is applied to imaged tissue (e.g., in image 502).

In some embodiments, the feedback component 214 may determine that the remedial action is automatic adjustment of an operational parameter to address the detected anomaly. For example, if too little tissue stretching is observed as in image 502, the feedback component 214 may generate a command for the control subsystem 208 to increase the degree of inflation of the balloon 110. As another example, if too much tissue stretching is observed as in image 506, the feedback component 214 may generate a command for the control subsystem 208 to decrease the degree of inflation of the balloon 110. Illustratively, the changes to pressure (increases or decreases) may be determined as a change within a particular range of inflation metrics (e.g., change to a different, lower, or higher target pressure metric within a pressure range for a desired level of tissue alteration or resistance), or they may be determined as a switch to a different level of inflation on a ladder of inflation levels (e.g., where each inflation level has its own pressure metric target and/or range of pressures). For example, the model 212 may use information from the control subsystem 208 (e.g., inflation parameters, such as target pressure or pressure level for the balloon) to determine whether the degree of stretching or other tissue alteration is expected, or is indicative of a procedure anomaly. In a case of over-stretching or under stretching for a particular level, the remedial action can be to alter balloon pressure within a range for the current level or the change to a different level. If the current pressure is an anchoring pressure and the tissue appears to be excessively stretched, the remedial action can be to deflate the balloon, partly or completely, to prevent damage to the tissue.

An example of a control subsystem operative to provide different levels of sub-anchoring and anchoring pressures, switch balloon inflation state from any level to a different level, and change target pressure within a particular range of inflation metrics, is described in applicant's published PCT Patent Application WO2012/120492, the disclosure of which is hereby incorporated by reference and made part of this specification. Advantageously, such a control subsystem may provide a sub-anchoring pressure level having a pressure range of 20 to 50 millibars, and preferably of 30 to 50 millibars. Preferably, the control subsystem is operative to provide a range of target pressure metrics within the pressure level of 30-50 millibars.

In some embodiments, the feedback component 214 may determine that a message (e.g., an alert, instruction, recommendation or other notification) is to be presented regarding the remedial action, such as a message regarding the manner in which inflation is to be adjusted. For example, if too little tissue stretching is observed as in image 502, the feedback component 214 may generate a notification for the user interface subsystem 206 to present, such as a message to the health care professional to increase the degree of inflation of the balloon 110, or a message prompting the health care professional to confirm whether the balloon 110 is inflating as expected. As another example, if too much tissue stretching is observed as in image 506, the feedback component 214 may generate a notification for the user interface subsystem 206 to present, such as a message to the health care professional to decrease the degree of inflation of the balloon 110. In some embodiments, a message regarding a change to pressure (increase or decrease) may specify a change within a particular range of inflation metrics (e.g., change to a different target pressure metric within a pressure range for a desired level of tissue alteration or resistance), or may specify as a change to a different level of inflation on a ladder of inflation levels (e.g., where each inflation level has its own pressure metric target and/or range of pressures). Such messages regarding change of pressure to be performed by the health care professional may be generated such that they are compatible with the available controls and user interface options of the control subsystem 208, such as knobs/buttons allowing tuning the pressure within a pressure level and/or switching between different pressure levels. For example, the model 212 may use information from the control subsystem 208 (e.g., inflation parameters, such as target pressure or pressure level for the balloon) to determine whether the degree of stretching or other tissue alteration is expected, or is indicative of a procedure anomaly. In a case of too much or too little stretching for a particular level, the message can recommend altering balloon pressure with a range for the current level or switching to a different level. If the current pressure is an anchoring pressure and the tissue appears to be excessively stretched, an alert or message may be presented to instruct the health care professional not to withdraw the balloon endoscope due to the current anchoring pressure.

FIG. 6 shows example procedure anomalies in which the internal imaging device is retracted too quickly (e.g., in example 626) and in which the internal imaging device is not retracted quickly enough (e.g., in example 622).

In some embodiments, the feedback component 214 may determine that the remedial action is automatic adjustment of an operational parameter to address the detected anomaly. For example, if the internal imaging device is not being retracted quickly enough as in example 622, the feedback component 214 may generate a command for the control subsystem 208 to decrease the degree of inflation of the balloon 110 to reduce resistance that may be slowing down retraction. As another example, if the internal imaging device is being retracted too quickly as in example 626, the feedback component 214 may generate a command for the control subsystem 208 to increase the degree of inflation of the balloon 110 to increase resistance and slow down retraction.

In some embodiments, the feedback component 214 may determine that a message (e.g., an alert or other notification) is to be presented regarding the remedial action, such as a message regarding the manner in which withdrawal speed is to be adjusted. For example, if the internal imaging device is not being retracted quickly enough as in example 622, the feedback component 214 may generate a notification for the user interface subsystem 206 to present, such as a message to the health care professional to speed up retraction and/or reduce the degree of inflation of the balloon 110. As another example, if the internal imaging device is being retracted too quickly as in mage 626, the feedback component 214 may generate a notification for the user interface subsystem 206 to present, such as a message to the health care professional to decrease the speed of retraction or increase the degree of inflation of the balloon 110.

FIG. 7 shows an example of a procedure anomaly that is based on a tissue anomaly, as described above. In some embodiments, the feedback component 214 may determine that the remedial action is automatic adjustment of an operational parameter to address the detected anomaly. For example, if the tissue anomaly 400 has been detected but the endoscope 100 continues to be retracted at sub-anchoring pressure, the feedback component 214 may generate a command for the control subsystem 208 to increase the degree of inflation of the balloon 110 to anchoring pressure. This inflation to anchoring pressure and prevention of further withdrawal of endoscope 100 may be particularly advantageous in enabling detection of certain tissue anomalies, which if missed and be undetected may have a serious adverse effect on patient's health, such as if the tissue anomaly is a tumor, a serious bleeding, a perforation or tear in the intestinal tissue, or any other serious adverse condition of the tissue. It is appreciated that instead of balloon 110 of endoscope 100, a different mechanical enhancement element having an anchoring state may be used, and be switched to anchoring state following a command generated by feedback component 214 as a remedial action to a tissue anomaly related procedure anomaly detected by analysis component 210 and model 212.

In some embodiments, the feedback component 214 may determine that a message (e.g., an alert or other notification) is to be presented regarding a remedial action to be taken. For example, the feedback component 214 may generate a notification for the user interface subsystem 206 to present, such as a message to the health care professional regarding detection of the tissue anomaly and/or a message to stop withdrawal and evaluate the tissue anomaly. This alert notification to stop further withdrawal of endoscope 100 and evaluate the tissue may be particularly advantageous in enabling detection of certain tissue anomalies, which if missed and be undetected may have a serious adverse effect on patient's health, such as if the tissue anomaly is a tumor, a serious bleeding, a perforation or tear in the intestinal tissue, or any other serious adverse condition of the tissue.

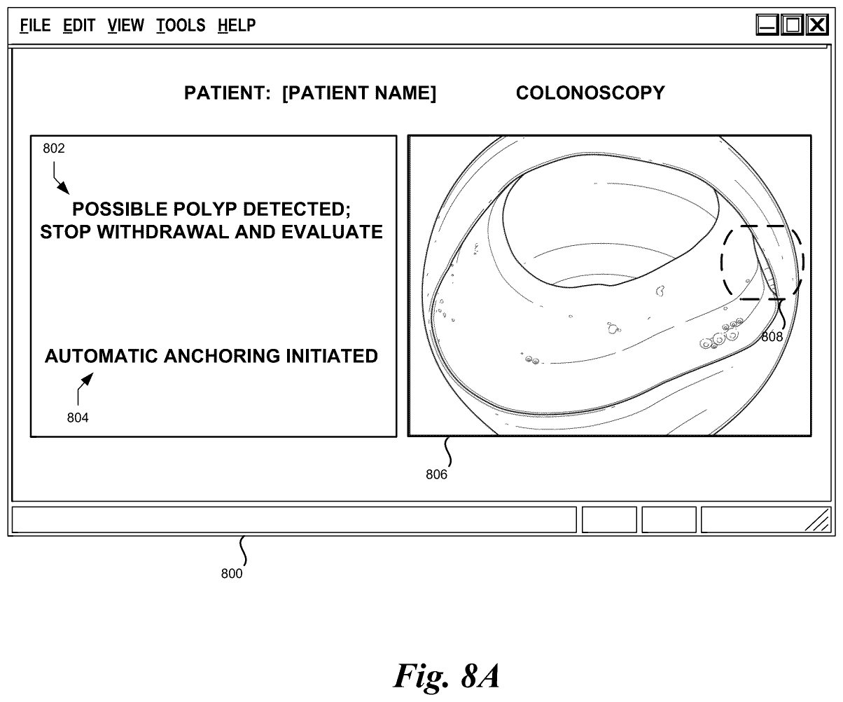

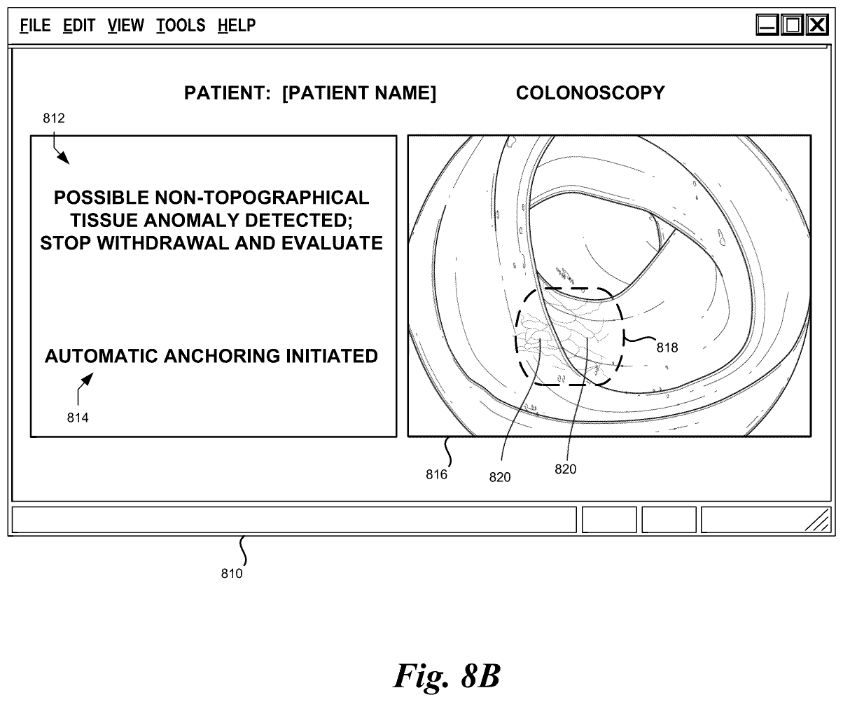

FIG. 8A illustrates an example user interface 800 that may present images and/or information to a health care professional during an internal imaging procedure. The user interface 800 may be generated or otherwise managed by the user interface subsystem 206, and may be presented on a monitor 104 or other display component of the internal imaging system.

In some embodiments, various notifications may be presented on the user interface 800. Returning to the example described above with reference to FIG. 7, in response to detection of a tissue anomaly, the user interface subsystem 206 may cause presentation of a message 802 indicating detection of the tissue anomaly and a recommended action to be taken by the health care professional. Additionally, or alternatively, if the remedial action(s) determined by the feedback component 214 includes a command for the control subsystem 208 to increase inflation of the balloon 110 to anchoring pressure, then the user interface subsystem 206 may cause presentation of a message such as message 804 to alert the health care professional to the action automatically taken or about to be taken.

In some embodiments, as shown, the interface 800 may include an image portion 806 for presentation of images captured by the visualization element of the internal imaging device. The images may be real-time or substantially real-time images so that the health care professional can accurately navigate the internal lumen in which the internal imaging device is located, and see tissue anomalies and other regions of interest. If a tissue anomaly is detected (e.g., using a tissue analysis model), a visual indication 808 may be presented to direct the health care professional's attention to the area of the image in which the tissue anomaly has been detected.

FIG. 8B illustrates detection of—and feedback regarding—a non-topographical tissue anomaly. A user interface 810 is depicted as displaying an image of non-topographical features of a patient's tissue in image portion 816. In the illustrated example, the non-topographical features are blood vessels 820 that have become more apparent through use of mechanical enhancement of the tissue. A possible tissue anomaly may have been detected (e.g., using a tissue analysis model) in the image of the non-topographical features. Based on detection of the of the possible tissue anomaly and, in some cases, based on detection that the endoscope 100 continues to be retracted, the dynamic procedure management subsystem 204 may trigger detection of a tissue-anomaly-related procedure anomaly and the user interface subsystem 206 may cause display of a visual indication 818 to direct the health care professional's attention to the area of the image in which the tissue anomaly has been detected. The user interface subsystem 206 may also, or alternatively, cause presentation of a message 812 indicating detection of the non-topographical tissue anomaly and a recommended action to be taken by the health care professional. Additionally, or alternatively, if the remedial action(s) determined by the feedback component 214 include(s) a command for the control subsystem 208 to increase inflation of the balloon 110 to anchoring pressure, then the user interface subsystem 206 may cause presentation of a message such as message 814 to alert the health care professional to the action automatically taken or about to be taken.

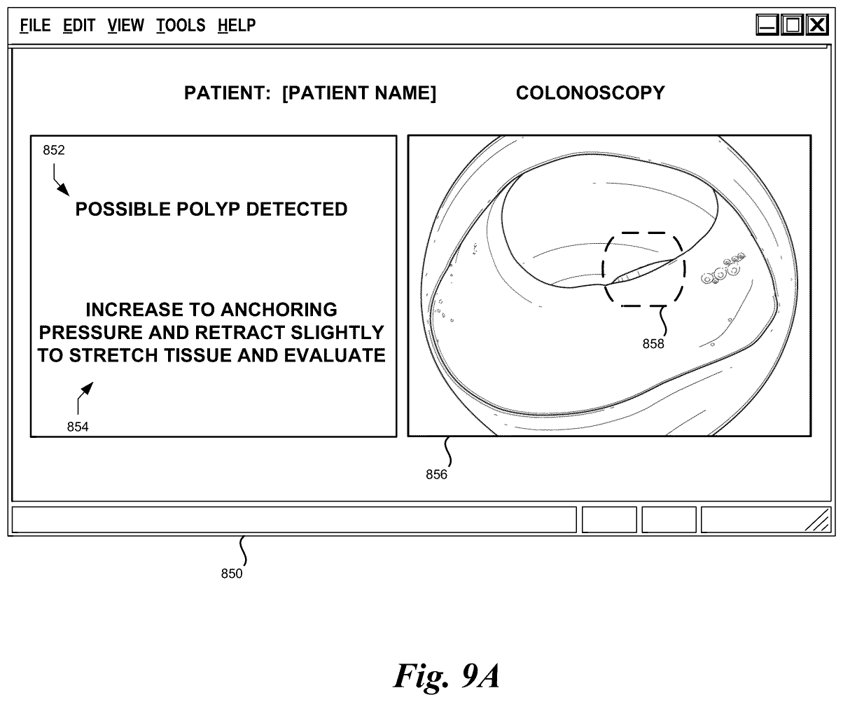

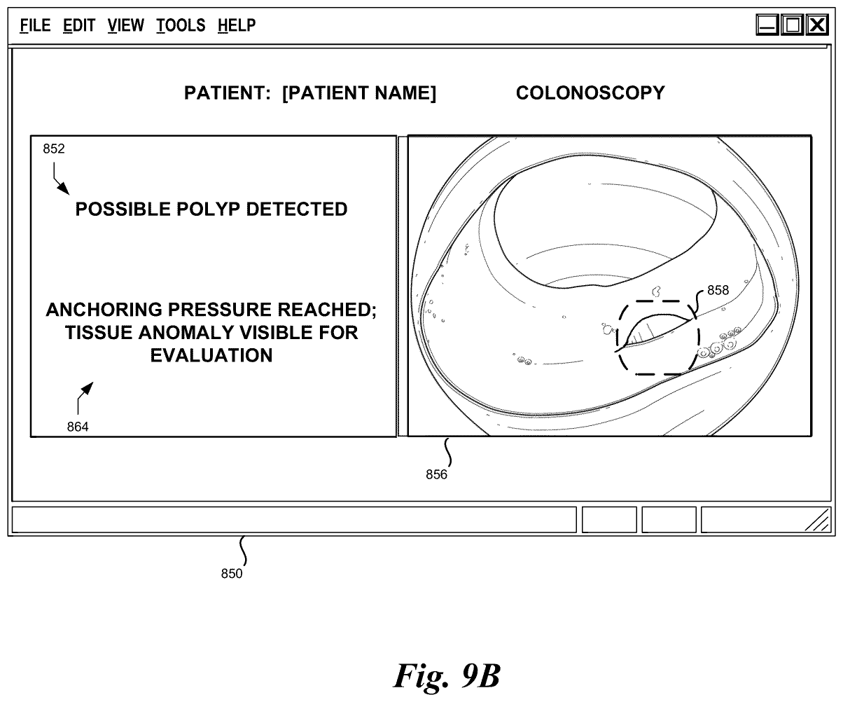

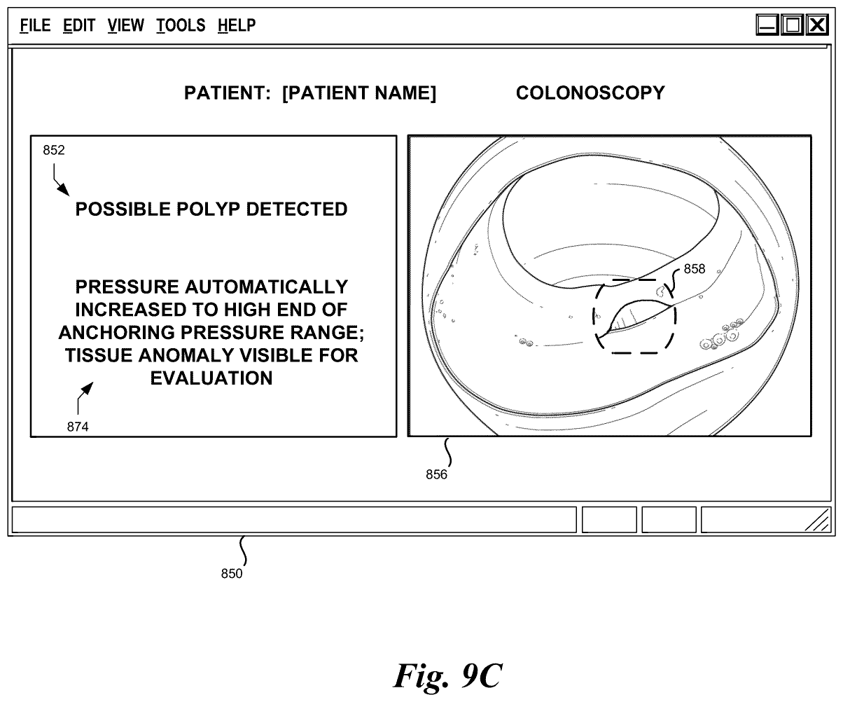

FIG. 9A illustrates detection of another tissue-anomaly-related procedure anomaly and presentation of feedback regarding a remedial action to be taken. In the illustrated example, a tissue anomaly (e.g., a polyp) is shown in the distance, just over a ridge or flexure of a patient's colon. The possible tissue anomaly may have been detected using a tissue analysis model. Based on detection of the possible tissue anomaly and, in some cases, based on detection that the endoscope 100 continues to be retracted and the tissue anomaly will eventually disappear from the field of view, or that the possible tissue anomaly is partially obscured and/or masked by the intestinal topography (e.g., by a ridge, flexure, fold or the like), the dynamic procedure management subsystem 204 may trigger detection of a tissue-anomaly-related procedure anomaly and the user interface subsystem 206 may cause display of a visual indication 858 to direct the health care professional's attention to the area of the image in which the tissue anomaly has been detected. The user interface subsystem 206 may also, or alternatively, cause presentation of a message 852 indicating detection of the tissue anomaly.

In the example illustrated in FIG. 9A, the feedback component 214 may have determined that the remedial action to be taken in response to the detected tissue-anomaly-related procedure anomaly is an increase in inflation of the balloon 110 to anchoring pressure and slight retraction of endoscope 100. A slight retraction of the endoscope 100 with the balloon 110 inflated to anchoring pressure can cause an additional degree of stretching of the tissue, beyond the degree to which the tissue is stretched with the balloon 110 inflated to a sub-anchoring pressure. With the additional degree of stretching, the tissue anomaly can be pulled along with the underlying tissue, which can have a visual effect of bringing the tissue anomaly around an obstruction (e.g., a ridge or flexure in a colon) and/or reducing the degree to which the obstruction obscures the tissue anomaly. In this case, the interface subsystem 206 may cause presentation of a message such as message 854 to alert the health care professional to the action to be taken.

FIG. 9B illustrates the effect of slight retraction of the endoscope 100 with the balloon 110 at an anchoring pressure after the detection and message shown in FIG. 9A and described above. As shown in the image portion 856 of the user interface 850 in FIG. 9B, the polyp indicated by visual indication 858 is no longer obstructed by the ridge or flexure of the colon. Instead, because the tissue has been stretched with the balloon at an anchoring pressure, the polyp is visible in a flatter area. In some embodiments, a confirmatory message 864 may be presented in response to detection. For example, the dynamic procedure management subsystem 204 may detect the stretching in the image of the tissue, along with the balloon being at an anchoring pressure.

FIG. 9C illustrates automatic implementation of a remedial action after a detection such as that shown in FIG. 9A and described above. Rather than instructing the health care professional to retract the endoscope 100 slightly with the balloon 110 at an anchoring pressure, the feedback component 214 may generate a command for the control subsystem 208 to increase inflation of the balloon 110 to an anchoring pressure. In some embodiments, the anchoring pressure used in such cases may be a higher pressure—that is still within a safe range of anchoring pressures—than the anchoring pressures used in other scenarios. When the balloon 110 is inflated to a comparatively high anchoring pressure, the outward force exerted on the tissue (e.g., the walls of the colon) may cause stretching that is similar to that described above with respect to FIG. 9B. For example, the stretching can serve to center a detected polyp, and mitigate or remove the effect of a flexure, ridge, or other obstruction. Thus, the health care professional's view of the polyp can be automatically enhanced by the command sent to and executed by the control subsystem 208. In addition, a message 874 can be presented to alert the health care professional to the remedial action that has been taken or will be taken.

In some embodiments, as shown in FIG. 9C, the exact visual effect may be different in some respects from the visual effect of retracting the endoscope 100 with the balloon 110 at anchoring pressure. As shown, the polyp indicated by the visual indication 858 in FIG. 9C has not been pulled quite as far from its original position as the polyp indicated by the visual indication 858 in FIG. 9B. This effect is due to the difference in tissue stretching realized by exclusive use of outward balloon pressure in FIG. 9C compared with the addition of retraction-based stretching in FIG. 9B.

It is appreciated that such change and tunning of the target anchoring pressure within an anchoring pressure range may facilitate better access to a potential tissue anomaly to be evaluated and/or treated and/or removed, by removing/reducing obstruction, bringing the potential tissue anomaly toward the center of the image, increasing or reducing the distance of the endoscope tip from the potential tissue anomaly to an optimized or otherwise desired distance, or other form of mechanical manipulation of the colon lumen to assist better access to the potential tissue anomaly. It is further appreciated that such mechanical manipulation through balloon pressure tunning can be performed automatically by the dynamic procedure management subsystem 204, which may provide pressure change instruction to control subsystem 208 in response to feedback from feedback component 214, without involvement of the health care professional. In some cases where endoscopic evaluation and/or intervention (e.g., polyp removal, clipping or coagulation of a bleeding blood vessel, etc.) is needed, the effect of change in balloon pressure, and particularly the change of the target anchoring pressure within an anchoring pressure range, may depend on specific conditions such as degree of deflection of the endoscope's tip, the curvature, cross-sectional diameter and other characteristics of the colon lumen at the vicinity of the potential tissue anomaly, and the like. Accordingly, dynamic procedure management subsystem 204 may tune the balloon pressure in an iterative process, effecting an incremental change in pressure, receiving feedback from feedback component 214, further changing the pressure according to the feedback, and repeating this sequence iteratively until the result (e.g., position of the tissue anomaly in the image, tip separation from the image, etc.) is satisfactory, or until no more improvement is achieved through pressure change. Such actions can be performed once the tissue anomaly was detected and prior to initiating the intervention (e.g., polyp removal, clipping or coagulation of a bleeding location, etc.), or can be performed in real-time during intervention, thereby providing optimized or otherwise desired positioning of the tissue anomaly under evaluation, treatment or removal.

Returning to FIG. 3, at decision block 314 the dynamic procedure management system 204 can determine whether the internal imaging procedure has ended. If the procedure is continuing, the routine 300 may return to block 304 to analyze additional images. Otherwise, the routine 300 may terminate at block 316.

In some embodiments, the dynamic procedure management system 204 may analyze subsequent images and verify that a remedial action was successful in resolving a procedure anomaly. For example, after an iteration of the routine 300 included detection of a procedure anomaly and generation of a message and/or command to alter an operational parameter, data may be stored or flag may be set to indicate detection of the procedure anomaly. A subsequent iteration of the routine 300 may not detect the procedure anomaly. In this case, a message may be generated for presentation via a user interface to confirm remediation of the procedure anomaly.

The dynamic procedure management system 204 may address procedure anomalies in addition to, or otherwise different from, the procedure anomalies described above.

In some embodiments, the analysis component 210 and model 212 may be configured to detect when a movable portion of the internal imaging device (e.g., the bending section and/or the tip of the balloon endoscope, including the visualization element) has been manipulated or otherwise deflected from a straight-ahead or otherwise default orientation. For example, if a first reference point (e.g., on a wall of a lumen) appears to cross a second reference point (e.g., a center point of an image), then a deflection event may be detected. The analysis component 210 may be configured to track the quantity of such deflection events over the course of an internal imaging procedure and/or over a sliding window of time. This data may be represented as a metric, such as a deflection rate. If the deflection rate exceeds a threshold, then a deflection rate anomaly may be detected and a remedial action may be determined.

In some embodiments, the analysis component 210 and model 212 may be configured to detect when a perforation of the tissue, a major bleeding, or any other life-threatening or otherwise adverse condition finding is depicted in an image. If such a perforation, major bleeding or other adverse condition finding is detected, then a remedial action may be determined. For example, the feedback component 214 may generate a command for the control subsystem 208 to deflate the balloon 110 of a balloon endoscope 100. As another example, the feedback component 214 may cause the user interface subsystem 206 to present an alert or other message to stop withdrawal of the endoscope and/or deflate the balloon. It is appreciated that the remedial action of deflating the balloon, whether by command to the control subsystem to automatically deflate the balloon or by instruction to the health care professional to manually deflate the balloon, may be advantageous in relieving excess pressure and/or stretching of the perforated or bleeding tissue or otherwise adverse condition finding, thereby preventing potential enlargement of the perforation, increase in bleeding, deterioration of an adverse condition, and/or further damage to the tissue. It is further appreciated that in case that the health care professional continues to withdraw the internal imaging device following detection of perforation or major bleeding by analysis component 210 and model 212, either prior to, during, or following balloon deflation, such continued withdrawal notwithstanding the presence of a perforation, major bleeding or other adverse condition may be detected by analysis component 210 and model 212 and classified as a procedure anomaly, to which feedback component 214 may generate a command for the control subsystem 208 to inflate the balloon 110 of a balloon endoscope 100 to anchoring pressure, as described above with reference to FIG. 7, in order to prevent further withdrawal of balloon endoscope 100 and disappearance of the perforation, major bleeding or other adverse condition finding from the field of view of visualization element 101.

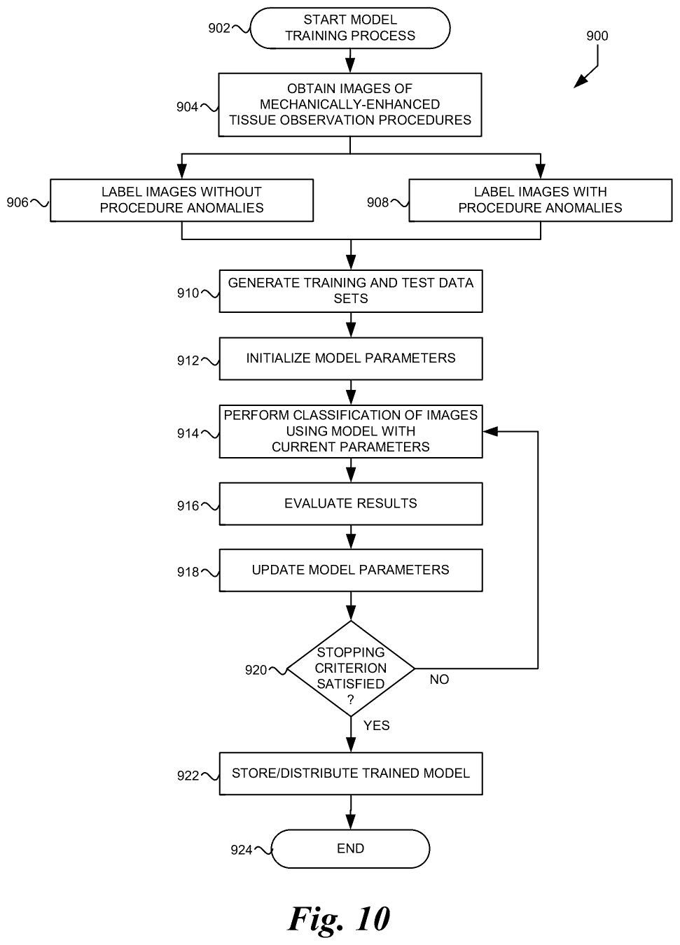

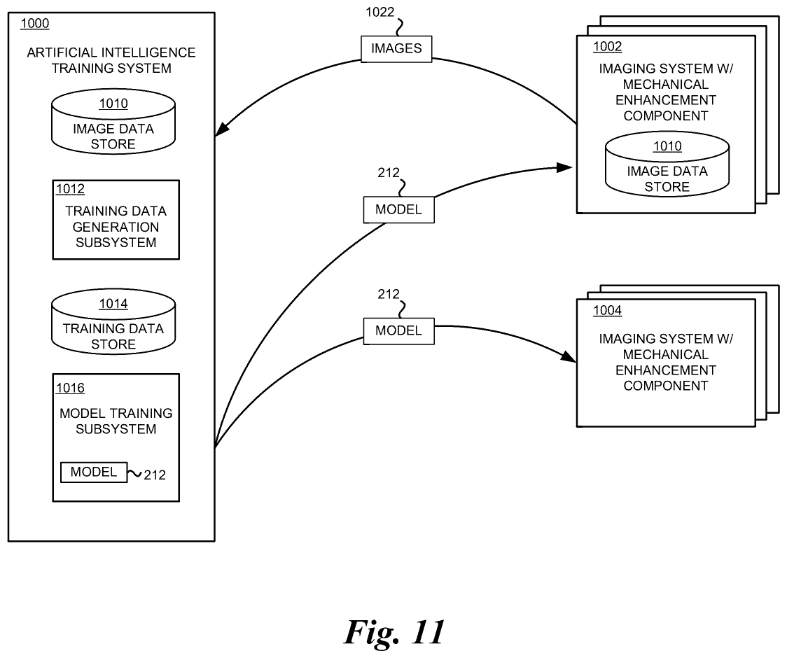

FIG. 10 is a flow diagram of an illustrative routine 900 that may be executed to train a mechanically-enhanced internal imaging procedure anomaly detection model 212 to generate detection output. Portions of the routine 900 will be described with further reference to the illustrative machine learning model 212 shown in FIG. 11.

Advantageously, the routine 900 generates training data using an internal imaging device with a mechanical enhancement element, such as a balloon endoscope. In some embodiments, the training data is generated using images depicting optimal or otherwise desired use of such an imaging device and the resulting mechanically-enhanced tissue. The training data may also be generated using images depicting various procedure anomalies, including those resulting in non-optimal or otherwise undesirable degrees of mechanical enhancement of tissue. By training a machine learning model using such images, the trained machine learning model incorporates and uses features of procedure anomalies that may interfere with analysis of mechanically-enhanced tissue, such as analysis using a tissue analysis model trained using images of mechanically-enhanced tissue to detect tissue anomalies. For example, if the tissue in an internal imaging procedure is not mechanically enhanced to the optimal or otherwise desired degree, the results of analyzing images of the tissue using such a tissue anomaly detection model may not be acceptable.

Although the routine 900 will be described with reference to training a machine learning model using images, it will be appreciated that training may also or alternatively be performed using video. For example, individual frames of video may be handled substantially as described with respect to images. Moreover, when the trained machine learning model is deployed for use in endoscope systems or other imaging systems, the input may be in the form of video, individual frames of which may be handled substantially as described with respect to images.

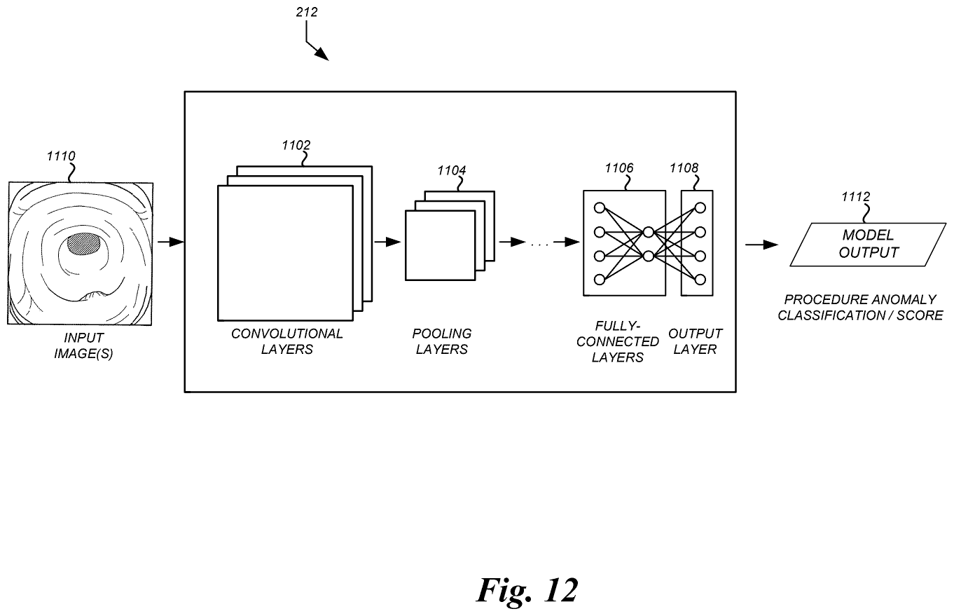

Portions of the routine 900 will be described with further reference to the illustrative data flows and interactions between components of the artificial intelligence training system 1000 and internal imaging systems 1002 and 1004 shown in FIG. 11. Additional portions of the routine 900 will be described with further reference to the illustrative machine learning model 212 shown in FIG. 12.

The routine 900 begins at block 902. The routine 900 may begin in response to an event, such as when an artificial intelligence training system 1000 begins operation, or in response to some other event or trigger. When the routine 900 is initiated, a set of executable program instructions stored on one or more non-transitory computer-readable media (e.g., hard drive, flash memory, removable media, etc.) may be loaded into memory (e.g., random access memory or “RAM”) of a computing device of the artificial intelligence training system, such as the Al training system computing device 1200 shown in FIG. 13 and described in greater detail below. In some embodiments, the routine 900 or portions thereof may be implemented on multiple processors, serially or in parallel.

At block 904, the artificial intelligence training system 1000 (also referred to herein simply as the “training system” for convenience) may obtain images from mechanically-enhanced tissue observation procedures from which to generate training data.

As shown in FIG. 11, the training system 1000 may include various subsystems and data stores to provide machine learning model training functionality. For example, the training system 1000 may include an image data store 1010 to store images generated using internal imaging devices with mechanical enhancement elements. The training system 1000 may also include a training data generation subsystem 1012 to label images and use the labelled images to generate training data, and a training data store 1014 to store training data. The training system 1000 may also include a model training subsystem 1016 for training a machine learning model 212 using training data from the training data store 1014.

In some embodiments, the training system 1000 (or individual components thereof) may be implemented on one or more host devices, such as blade servers, midrange computing devices, mainframe computers, desktop computers, or any other computing device configured to provide computing services and resources. For example, a single host device may execute one or more image data stores 1010, training data generation subsystems 1012, training data stores 1014, model training subsystems 1016, some combination thereof, etc. The training system 1000 may include any number of such hosts.

In some embodiments, the features and services provided by the training system 1000 may be implemented as web services consumable via one or more communication networks. In further embodiments, the training system 1000 (or individual components thereof) is provided by one or more virtual machines implemented in a hosted computing environment. The hosted computing environment may include one or more rapidly provisioned and released computing resources, such as computing devices, networking devices, and/or storage devices. A hosted computing environment may also be referred to as a “cloud” computing environment.

The training system 1000 may obtain images 1022 from one or more imaging systems 1002. The imaging systems 1002 may include any of a variety of imaging systems that have mechanical enhancement elements configured to apply a mechanical enhancement to tissue for imaging. In some embodiments, an imaging system 1002 may be an endoscope system 102 that includes or is in communication with an endoscope 100 with a selectively inflatable/deflatable balloon 110, as shown in FIG. 1. The endoscope 100 may be inserted into a cavity of a patient and advanced to a location for imaging, such as an intestinal lumen (e.g., the interior of the patient's colon). As described in great detail above, the balloon 110 may be selectively inflated to a sub-anchoring pressure, and retracted. Advantageously, retraction of the endoscope 100 with the balloon 110 inflated at sub-anchoring pressure stretches the interior tissue of the intestinal lumen, and images of the stretched tissue may be taken (e.g., still images, video, or a combination thereof) using a visualization element such as a CCD 101.

Images taken of the mechanically-enhanced (stretched) tissue can include images of tissue that is mechanically enhanced to an optimal or otherwise desired degree so that the model 212 can be trained to determine when such desired conditions are present. For example, a highly skilled health care professional may perform procedures during which images are taken, and those images may be considered images of normal procedure conditions that do not depict procedure anomalies. Images may also be taken of tissue that is mechanically enhanced to a non-optimal or otherwise undesirable degree so that the model 212 can be trained to determine when such undesirable conditions are present. In some embodiments, images may be taken of tissue that is not mechanically enhanced so that the model 212 can be trained to determine when such conditions are present.

Although certain examples described herein refer to an endoscope with a balloon-based mechanical enhancement element, the examples are illustrative only, and are not intended to be limiting. In some embodiments, other imaging systems and/or mechanical enhancement elements be used uniformly or in various combinations.

The imaging systems 1002 may send images 1022 to the training system 1000 as the images are generated (e.g., during imaging procedures), after imaging procedures (e.g., in a batch), on demand after a request from the training system 1000, on a schedule, or in response to some other event. The training system 1000 may store the images 1022 in an images data store 1010. In some embodiments, all images may be obtained from a single imaging system 1002 rather than a set of multiple imaging systems 1002.

In some embodiments, images 1022 may be pre-processed prior to, or as part of the process of, generating training data upon which to train a machine learning model. For example, the resolution of images may be standardized to a resolution upon which the machine learning model is configured to operate (e.g., based on the size of various layers of the model 212). As another example, images may be segmented into smaller portions for process instead of, or in addition to, using entire images from imaging systems 1002.

At block 906, the training data generation subsystem 1012 may label a portion of the images 1022 that do not include a procedure anomaly to be detected by the machine learning model 212. In some embodiments, a subset of the images 1022 may have been previously tagged as being negative for the presence of a procedure anomaly. For example, during or after the process of generating images, a user of an imaging system 1002 may indicate images that are negative for the presence of a procedure anomaly. Tag data may be incorporated into such images, or provided to the training system 1002 as metadata separately from the images. The tag data may include a flag or other indicator of whether there is no procedure anomaly in the corresponding image. The training data generation subsystem 1012 may access the tag data and, based thereon, label a portion of the images 1022 as not including a procedure anomaly. The labeled images may be stored as training data in the training data store 1014.

In some embodiments, a portion of the images 1022 may not have been previously tagged as being negative for the presence of a procedure anomaly. For such images, the training data generation subsystem 1012 may generate or otherwise obtain labels for those images 1022 that are negative for the presence of a procedure anomaly. For example, the training data generation subsystem 1012 may provide a user interface for healthcare professionals or other experts. The user interface may be a graphical user interface delivered as a web page, mobile application interface, desktop application interface, or via some other mechanism of delivery. Users may use the interface to view images and indicate one or more of: which images do and/or do not include procedure anomalies; where any procedure anomalies are located within individual images; more detailed information regarding the procedure anomalies (e.g., whether they are associated with excessively altered tissue, inadequately altered tissue, excessive withdrawal speed, inadequate withdrawal speed, presence of a tissue anomaly without stopping to evaluate the tissue anomaly, too many image device manipulations, or some other specific procedure anomaly), etc. Interactions to indicate the presence or absence of procedure anomalies (or other associated information) can be used to generate tag data that may be incorporated into the images, or provided to the training system 1002 as metadata separately from the images. The tag data may include a flag or other indicator of whether there is no procedure anomaly in the corresponding image. The training data generation subsystem 1012 may access the tag data and, based thereon, label a portion of the images 1022 as not including a procedure anomaly. The labeled images may be stored as training data in the training data store 1014.

At block 908, the training data generation subsystem 1012 may label a portion of the images 1022 that include a procedure anomaly to be detected by the machine learning model 212. In some embodiments, a portion of the images 1022 may have been previously tagged as being positive for the presence of a procedure anomaly. For example, during or after the process of generating images, a user of an imaging system 1002 may indicate images that are positive for the presence of a procedure anomaly. Tag data may be incorporated into such images, or provided to the training system 1002 as metadata separately from the images. The tag data may include a flag or other indicator of whether there is any procedure anomaly in the corresponding image, where in the image the procedure anomaly may be located, additional information regarding the nature of the procedure anomaly (e.g., whether it is associated with excessively altered tissue, inadequately altered tissue, excessive withdrawal speed, inadequate withdrawal speed, presence of a tissue anomaly without stopping to evaluate the tissue anomaly, too many image device manipulations, or some other specific procedure anomaly), etc. The training data generation subsystem 1012 may access the tag data and, based thereon, label a portion of the images 1022 as including a procedure anomaly, and optionally which portion of individual images are indicative of the procedure anomaly. Illustratively, labelling of an image to indicate a procedure anomaly may include generating labelling data from the tag data, or copying the tag data, to indicate a coordinate location of a procedure anomaly, an offset from a reference location of a procedure anomaly, a range of locations for a procedure anomaly, or some other data from which the training system 1000 can train the machine learning model 212 to detect procedure anomalies in an image. The labeled images may be stored as training data images in the training data store 1014.

In some embodiments, a portion of the images 1022 may not have been previously tagged as being positive for the presence of a procedure anomaly. For such images, the training data generation subsystem 1012 may generate or otherwise obtain labels for those images 1022 that are positive for the presence of a procedure anomaly. For example, as described above with respect to images that are negative for the presence of a procedure anomaly, the training data generation subsystem 1012 may provide a user interface for healthcare professionals or other experts to view images and indicate one or more of: which images do and/or do not include procedure anomalies; which portions of individual images are indicative of procedure anomalies; more detailed information regarding the procedure anomalies (e.g., whether it is associated with excessively altered tissue, inadequately altered tissue, excessive withdrawal speed, inadequate withdrawal speed, presence of a tissue anomaly without stopping to evaluate the tissue anomaly, too many image device manipulations, or some other specific procedure anomaly), etc. Interactions to indicate the presence or absence of procedure anomalies (or other associated information) can be used to generate tag data that may include a flag or other indicator of whether there is a procedure anomaly in the corresponding image. The training data generation subsystem 1012 may access the tag data and, based thereon, label a portion of the images 1022 as including a procedure anomaly, the nature of the procedure anomaly, etc. The labeled images may be stored as training data images in the training data store 1014.

Although blocks 906 and 908 are shown as separate blocks in parallel paths of execution, the illustration is an example only and is not intended to be limiting. In some embodiments, operations associated with blocks 906 and 908 may be performed serially, with one block occurring before the other. In some embodiments, the operations associated with blocks 906 and 908 may be performed in one step, during which images are analyzed, some images are labelled as negative for procedure anomalies, and others are labelled as positive for a procedure anomaly, without regard to the order in which the respective images are processed.

At block 910, the training data generation subsystem 1012 or some other subsystem of the training system 1000 may select training data to be used during the current instance of the routine 900 to train the machine learning model 212. In some embodiments, the training data generation subsystem 1012 may separate the labelled training images in the training data store 1014 into a training set and a testing set. The training set may be used as described in greater detail below to train the machine learning model 212. The testing set may be used to test the trained machine learning model 212. Advantageously, using a separate testing set of images to test the performance of the machine learning model 212 can help to determine whether the trained machine learning model 212 can generalize the training to new images that were not presented to the machine learning model during training (or during an iteration of testing).

At block 912, the model training subsystem 1016 can initialize the parameters of the machine learning model 212 to be trained. In some embodiments, the machine learning model may be implemented as a neural network (“NN”).

Generally described, NNs—including deep neural networks (“DNNs”), convolutional neural networks (“CNNs”), recurrent neural networks (“RNNs”), other NNs, and combinations thereof—have multiple layers of nodes, also referred to as “neurons.” Illustratively, a NN may include an input layer, an output layer, and any number of intermediate, internal, or “hidden” layers between the input and output layers. The individual layers may include any number of separate nodes. Nodes of adjacent layers may be logically connected to each other, and each logical connection between the various nodes of adjacent layers may be associated with a respective weight. Conceptually, a node may be thought of as a computational unit that computes an output value as a function of a plurality of different input values. Nodes may be considered to be “connected” when the input values to the function associated with a current node include the output of functions associated with nodes in a previous layer, multiplied by weights associated with the individual “connections” between the current node and the nodes in the previous layer. When a NN is used to process input data in the form of an input vector or a matrix of input vectors (e.g., data representing an image, such as the values of the individual pixels of the image), the NN may perform a “forward pass” to generate an output vector or a matrix of output vectors, respectively. The input vectors may each include n separate data elements or “dimensions,” corresponding to the n nodes of the NN input layer (where n is some positive integer, such as the total number of pixels in an input image). Each data element may be a value, such as a floating-point number or integer (e.g., a greyscale value or a red-blue-green or “RBG” value of a pixel). A forward pass typically includes multiplying input vectors by a matrix representing the weights associated with connections between the nodes of the input layer and nodes of the next layer, applying a bias term, and applying an activation function to the results. The process is then repeated for each subsequent NN layer. Some NNs have hundreds of thousands or millions of nodes, and millions of weights for connections between the nodes of all of the adjacent layers.

The trainable parameters of the NN include the weights (and in some embodiments the bias terms) for each layer that are applied during a forward pass. In some embodiments, to initialize the parameters of the machine learning model, the model training subsystem 1016 can use a pseudo-random number generator to assign pseudo-random values to the parameters. In some embodiments, the parameters may be initialized using other methods. For example, a machine learning model 212 that was previously trained using the routine 900 or some other process may serve as the starting point for the current iteration of the routine 900.

At block 914, the model training subsystem 1016 can analyze training data images using the model 212 to produce training data output. Illustratively, the training data output may correspond to classification determinations regarding whether training data images are negative or positive for procedure anomalies, which portions of the images are likely to be negative or positive, and/or the nature of the procedure anomalies. In subsequent blocks of the routine 900, the training data output is used to evaluate the performance of the model 212 and apply updates to the trainable parameters.

With reference to FIG. 12, the structure and operation of illustrative embodiment of a machine learning model 212 to generate training data output (and, similarly, prediction output in production implementations of the trained machine learning model 212) will be described. The illustrative machine learning model 212—also referred to simply as a “model” for convenience—is implemented as a CNN. As shown, the model 212 includes one or more convolutional layers 1102, one or more max pooling layers 1104, and a set of fully-connected layers 1106 before an output layer 1108. The convolutional layers 1102 and max pooling layers 1104 are used to iteratively “convolve” (e.g., use a sliding window to process portions of) an input image 1110 and determine a degree to which a particular “feature” (e.g., an edge or other aspect of an object to be detected) is present in different portions of the input image 1110. Aspects of this procedure may also be referred to as “feature mapping.” The procedure may be performed using any number of sets of convolutional layers 1102 and max pooling layers 1104 (e.g., 1, 2, 5, 10, or more sets). The result that is generated by the sets of convolutional layers 1102 and max pooling layers 1104 may be a matrix of numbers, such as floating-point numbers. The matrix may then be converted to a vector for processing by the set of fully-connected layers 1106. The fully-connected layers 1106 can generate classification output indicating whether the input image 1110 is positive or negative for a procedure anomaly. For example, a particular output value or set of output values may represent a classification as positive or negative (e.g., a value>=0.5 indicates a positive classification, a value <0.5 indicates a negative classification). In some embodiments, the output of the fully-connected layers 1106, or separate output generated by or otherwise derived from output generated by the convolutional layers 1102 and max pooling layers 1104, can indicate the location(s) within the input image 1110 that are indicative of a procedure anomaly, the nature of the procedure anomaly, etc.

An example of the processing performed by the model 212 will now be described with reference first to the operation of the fully-connected layers 1106 at the end of the model 212 and then to the convolutional and max pooling layers 1102 and 1104 at the beginning of the model 212. The set of fully-connected layers 1106 may include an input layer by which output of the convolutional layer(s) 1102 and max pooling layer(s) 1104 is received. The set of fully-connected layers 1106 includes the input layer with a plurality of nodes, one or more internal layers each with a plurality of nodes, and an output layer with a plurality of nodes. The specific number of layers shown in FIG. 12 is illustrative only, and is not intended to be limiting. In some models 212, the set of fully-connected layers 1106 may include different numbers of internal layers and/or different numbers of nodes in the input, internal, and/or output layers. For example, in some models 212 the layers may have hundreds or thousands of nodes. As another example, in some models 212 there may be 1, 2, 4, 5, 10, 50, or more internal layers. In some implementations, each layer may have the same number or different numbers of nodes. For example, the input layer or the output layer can each include more or less nodes than the internal layers. The input layer and the output layer can include the same number or different number of nodes as each other. The internal layers can include the same number or different numbers of nodes as each other.

The connections between individual nodes of adjacent layers of the set of fully-connected layers 1106 are each associated with a trainable parameter, such as a weight and/or bias term, that is applied to the value passed from the prior layer node to the activation function of the subsequent layer node. For example, the weights associated with the connections from the input layer to an internal layer to which it is connected may be arranged in a weight matrix W with a size m×n, where m denotes the number of nodes in an internal layer and n denotes the dimensionality of the input layer. The individual rows in the weight matrix W may correspond to the individual nodes in the input layer, and the individual columns in the weight matrix W may correspond to the individual nodes in the internal layer. The weight w associated with a connection from any node in the input layer to any node in the internal layer may be located at the corresponding intersection location in the weight matrix W.

Illustratively, a vector representing output of the convolutional layer(s) 1102 and max pooling layer(s) 1104 may be computed or otherwise obtained by a computer processor that stores or otherwise has access to the weight matrix W. The processor then multiplies the vector by the weight matrix W to produce an intermediary vector. The processor may adjust individual values in the intermediary vector using an offset or bias that is associated with the internal layer (e.g., by adding or subtracting a value separate from the weight that is applied). In addition, the processor may apply an activation function to the individual values in the intermediary vector (e.g., by using the individual values as input to a rectified linear unit (“ReLU”) function or a sigmoid function).

In some embodiments, there may be multiple internal layers, and each internal layer may or may not have the same number of nodes as each other internal layer. The weights associated with the connections from one internal layer (also referred to as the “preceding internal layer”) to the next internal layer (also referred to as the “subsequent internal layer”) may be arranged in a weight matrix similar to the weight matrix W, with a number of rows equal to the number of nodes in the subsequent internal layer and a number of columns equal to the number of nodes in the preceding internal layer. The weight matrix may be used to produce another intermediary vector using the process described above with respect to the input layer and first internal layer. The process of multiplying intermediary vectors by weight matrices and applying activation functions to the individual values in the resulting intermediary vectors may be performed for each internal layer of the fully-connected layers 1106 subsequent to the initial internal layer of the fully-connected layers 1106.

The output layer of the model 212 makes output determinations from the last intermediary vector. Weights associated with the connections from the last internal layer to the output layer may be arranged in a weight matrix similar to the weight matrix W, with a number of rows equal to the number of nodes in the output layer and a number of columns equal to the number of nodes in the last internal layer. The weight matrix may be used to produce an output vector 1108 using the process described above with respect to the input layer and first internal layer.

The output vector 1108 may include data representing the classification or regression determinations made by the model 212 for the input image 1110. Some models 212 are configured to make u classification determinations corresponding to u different classifications (where u is a number corresponding to the number of nodes in the output layer). The data in each of the u different dimensions of the output vector may be a confidence score indicating the probability that the input image 1110 is properly classified in a corresponding classification. Some models 212 are configured to generate values based on regression determinations rather than classification determinations, or regression determinations that correspond to classification determinations.

The training data from which the training images 1110 are drawn may also include reference data output vectors. Each reference data output vector may correspond to a training image 1110, and may include the “correct” or otherwise desired output that the model 212 should produce for the corresponding training image 1110. For example, a reference data output vector may include scores indicating the proper classification(s) for the corresponding training image 1110 (e.g., scores of 1.0 for the proper classification(s), and scores of 0.0 for improper classification(s)). As another example, a reference data output vector may include scores indicating the proper regression output(s) for the corresponding training data input vector. The goal of training may be to minimize the difference between the model output 1112 and corresponding reference data output vectors.

Prior to the set of fully-connected layers 1106, the image 1110 may be analyzed using one or more convolutional layers 1102 and one or more max pooling layers 1104. Like the set of fully-connected layers 1106, the convolutional layers 1102 are associated with trainable parameters (e.g., weights, biases) that are applied to portions of layer input, such as portions of the image 1110, portions of a prior convolutional layer 1102 output, or portions of a max pooling layer 1104 output. However, unlike the fully-connected layers 1106, the nodes in a convolutional layer 1102 may only be connected to a small region of the preceding layer instead of all of the neurons in a fully-connected manner.

By way of illustration, a training image 1110 may be represented as a matrix (e.g., for a greyscale image) or a tensor (e.g., for an RGB image with three color channels) of values in which individual values represent individual pixel values of the image 1110. A convolutional layer 1102 can generate layer output for nodes connected to particular regions in the input image 1110. For example, each node of a convolutional layer 1102 corresponds to a dot product of its associated weights and a region of the prior layer (or input image 1110). There may be more than one feature for which input is being assessed for detection, and the existence of each feature may be assessed using a separate “filter” represented by a set of weights. Thus, in some embodiments the output of a given convolutional layer 1102 may be represented as three-dimensional tensor with two dimensions corresponding to spatial dimensions of the input image 1110 and a third dimension corresponding to the number of filters. An activation function, such as ReLU, may also be applied elementwise to each node. These operations may be performed substantially as described above with respect to general NNs and the set of fully connected layers 1106, with adjustment for the limited connectivity of the convolutional layer. A max pooling layer 1104 may effectively perform a compression operation on the output of a preceding convolutional layer 1102 resulting in max pooling layer output that is reduced in spatial dimensions with respect to the size of the input image 1110.

A model 212 implemented as shown and described above thus transforms an input image 1110 from the image's pixel values to the final detection scores (e.g., classification or regression scores) output by the model 212. In doing so, the convolutional layers 1102 and fully connected layers 1106 perform transformations that are a function of not only their respective inputs (e.g., the inputs from prior layers), but also of the parameters of the layers (the weights and biases of the neurons). Other portions of the model 212 may not have separate trainable parameters. For example, the max pooling layers 1104 and any ReLU functions may implement fixed functions that depend only on their respective inputs and are not necessarily trainable.

Returning to the routine 900 shown in FIG. 10, at block 916 the model training subsystem 1016 can evaluate the results of processing one or more training input images 1110 using the model 212. In some embodiments, the model training subsystem 1016 may evaluate the results using a loss function, such as a binary cross entropy loss function, a weighted cross entropy loss function, a squared error loss function, a softmax loss function, some other loss function, or a composite of loss functions. The loss function can evaluate the degree to which trading data output vectors generated using the model 212 differ from the desired output (e.g., reference data output vectors) for corresponding training data images.

At block 918, the model training subsystem 1016 can update parameters of the model 212 based on evaluation of the results of processing one or more training input images 1110 using the model 212. The parameters may be updated so that if the same training data images are processed again, the output produced by the model 212 will be closer to the desired output represented by the reference data output vectors that correspond to the training data images. In some embodiments, the model training subsystem 1016 may compute a gradient based on differences between the training data output vectors and the reference data output vectors. For example, gradient (e.g., a derivative) of the loss function can be computed. The gradient can be used to determine the direction in which individual parameters of the model 212 are to be adjusted in order to improve the model output (e.g., to produce output that is closer to the correct or desired output for a given input). The degree to which individual parameters are adjusted may be predetermined or dynamically determined (e.g., based on the gradient and/or a hyper parameter). For example, a hyper parameter such as a learning rate may specify or be used to determine the magnitude of the adjustment to be applied to individual parameters of the model 212.

In some embodiments, the model training subsystem 1016 can compute the gradient for a subset of the training data, rather than the entire set of training data. Therefore, the gradient may be referred to as a “partial gradient” because it is not based on the entire corpus of training data. Instead, it is based on the differences between the training data output vectors and the reference data output vectors when processing only a particular subset of the training data.

With reference to an illustrative embodiment, the model training subsystem 1016 can update some or all parameters of the machine learning model 212 (e.g., the weights of the model) using a gradient descent method with back propagation. In back propagation, a training error is determined using a loss function (e.g., as described above). The training error may be used to update the individual parameters of the model 212 in order to reduce the training error. For example, a gradient may be computed for the loss function to determine how the weights in the weight matrices are to be adjusted to reduce the error. The adjustments may be propagated back through the model 212 layer-by-layer.

At decision block 920, the model training subsystem 1016 can in some embodiments determine whether one or more stopping criteria are met. For example, a stopping criterion can be based on the accuracy of the machine learning model 212 as determined using the loss function, the test set, or both. As another example, a stopping criterion can be based on the number of iterations (e.g., “epochs”) of training that have been performed, the elapsed training time, or the like. If the one or more stopping criteria are met, the routine 900 can proceed to block 922; otherwise, the routine 900 can return to block 914 or some other prior block of the routine 900.

At block 922, the model training subsystem 1016 can store and/or distribute the trained model 212. The routine 900 may terminate at block 924.

As shown in FIG. 11, the trained model 212 can be distributed to one or more imaging systems 1004 for use in imaging procedures. In some embodiments, the trained model 212 can additionally or alternatively be distributed to the imaging systems 1002 from which the training system 1000 obtained images 1022 for training the model 212.

In some embodiments, as described above, the imaging systems 1002 or 1004 may include an endoscope system 102 as shown in FIG. 1. The endoscope system 102 may include a computing device with one or more computer processors programmed by executable instructions to, among other things, process image data obtained from a visualization element of a balloon endoscope 100 (e.g., still images, video, etc.) and present the image data on a monitor 104. When supplied with the trained model 212, the endoscope system 102 can analyze the image data using the trained model 212, detect procedure anomalies, and implement remedial actions. For example, if output of the trained model 212 for a particular image or portion of video indicates a positive classification for the presence of a procedure anomaly, the presentation on the monitor 104 may be updated to indicate the positive classification, the type of procedure anomaly, etc. In some embodiments, output of the model 212 may be used to determine and implement changes to operational parameters via the control subsystem 208 in addition to, or instead of, presentations via the monitor 104, as described in greater detail above.

FIG. 13 illustrates an example training system computing device 1200 that may be used in some embodiments to execute the processes and implement the features of the training system 1000 described above. In some embodiments, the computing device 1200 may include: one or more computer processors 1202, such as physical central processing units (“CPUs”) or graphics processing units (“GPUs”); one or more network interfaces 1204, such as a network interface cards (“NICs”); one or more computer readable medium drives 1206, such as high density disks (“HDDs”), solid state drives (“SSDs”), flash drives, and/or other persistent non-transitory computer-readable media; and one or more computer readable memories 1210, such as random access memory (“RAM”) and/or other volatile non-transitory computer-readable media. The network interface 1204 can provide connectivity to one or more networks or computing devices. The computer processor 1202 can receive information and instructions from other computing devices or services via the network interface 1204. The network interface 1204 can also store data directly to the computer-readable memory 1210. The computer processor 1202 can communicate to and from the computer-readable memory 1210, execute instructions and process data in the computer readable memory 1210, etc.

The computer readable memory 1210 may include computer program instructions that the computer processor 1202 executes in order to implement one or more embodiments. The computer readable memory 1210 can store an operating system 1212 that provides computer program instructions for use by the computer processor 1202 in the general administration and operation of the computing device 1200. The computer readable memory 1210 can also include machine learning model training instructions 1214 for implementing training of machine learning models. The computer readable memory 1210 can further include computer program instructions and other data for implementing aspects of the present disclosure, such as the procedure analysis model 212 (or a portion thereof) that is being trained.

FIG. 13 also illustrates an example imaging system computing device 1250 that may be used in some embodiments to execute the processes and implement the features of the imaging systems 1002, 1004, etc. described above. The imaging system computing device 1250 may include components that are similar in some or all respects to components of the training system computing device 1200 described above. For example, the computing device 1250 may include: one or more computer processors 1252, one or more network interfaces 1254, one or more computer readable medium drives 1256, and one or more computer readable memories 1260. The computer readable memory 1260 may include computer program instructions that the computer processor 1252 executes in order to implement one or more embodiments. The computer readable memory 1260 can store an operating system 1262 that provides computer program instructions for use by the computer processor 1252 in the general administration and operation of the computing device 1250. The computer readable memory 1260 can also include imaging procedure management instructions 1264 for implementing an imaging procedure and analyzing images. The computer readable memory 1260 can further include computer program instructions and other data for implementing aspects of the present disclosure, such as a procure analysis model 212 with which the computing device 1250 analyzes images generated during an imaging procedure to detect procedure anomalies, and a tissue analysis model 1270 with which the computing device 1250 analyzes images to detect tissue anomalies.

Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in certain embodiments, operations or events can be performed concurrently, e.g., through multi-threaded processing, interrupt processing, or multiple processors or processor cores or on other parallel architectures, rather than sequentially.

The various illustrative logical blocks, modules, routines, and algorithm steps described in connection with the embodiments disclosed herein can be implemented as electronic hardware, or combinations of electronic hardware and computer software. To clearly illustrate this interchangeability, various illustrative components, blocks, modules, and steps have been described above generally in terms of their functionality. Whether such functionality is implemented as hardware, or as software that runs on hardware, depends upon the particular application and design constraints imposed on the overall system. The described functionality can be implemented in varying ways for each particular application, but such implementation decisions should not be interpreted as causing a departure from the scope of the disclosure.

Moreover, the various illustrative logical blocks and modules described in connection with the embodiments disclosed herein can be implemented or performed by a machine, such as a processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without other input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

1. A system for machine-learning-based analysis of an endoscopy procedure, the system comprising:
a balloon endoscope comprising a visualization element and an inflatable balloon, wherein the balloon endoscope is configured to mechanically enhance visualization of tissue when moved within an intestinal lumen of a patient with the inflatable balloon inflated to a sub-anchoring pressure, the inflatable balloon causing axial stretching of tissue of the intestinal lumen to at least partially flatten or unfold natural topography of the tissue; and
a computing device comprising one or more processors and computer-readable memory, the computing device programmed by executable instructions to at least:
obtain an image of a portion of the tissue using the visualization element;
analyze the image using a machine learning model trained to generate classification output data representing a procedure anomaly classification;
determine, based at least partly on the classification output data, that the image corresponds to a procedure anomaly;
generate feedback data based on the procedure anomaly, wherein the feedback data represents a remedial action to be taken with respect to the balloon endoscope; and
send the feedback data to at least one of a user interface subsystem or a control subsystem.

2. The system of claim 1, wherein the computing device is programmed by further executable instructions to:
determine, based at least partly on the procedure anomaly being one of an under-inflation anomaly or an over-inflation anomaly, that the remedial action comprises a change to an inflation pressure of the balloon; and
display, on a user interface, a message indicating a manner in which the inflation pressure is to be changed.

3. The system of claim 1, wherein the computing device is programmed by further executable instructions to:
determine, based at least partly on the procedure anomaly being one of a low withdrawal speed anomaly or a high withdrawal speed anomaly, that the remedial action comprises a change to a withdrawal speed of the balloon endoscope; and
display, on a user interface, a message indicating a manner in which the withdrawal speed is to be changed.

4. The system of claim 3, wherein the procedure anomaly comprises a low withdrawal speed anomaly and the manner in which the withdrawal speed is to be changed comprises an increase in the withdrawal speed.

5. The system of claim 3, wherein the procedure anomaly comprises a high withdrawal speed anomaly and the manner in which the withdrawal speed is to be changed comprises a reduction in the withdrawal speed.

6. The system of claim 1, wherein the computing device is programmed by further executable instructions to:
determine, based at least partly on the procedure anomaly comprising detection of a tissue anomaly, that the remedial action comprises stopping withdrawal of the balloon endoscope; and
display, on a user interface, a message indicating withdrawal of the balloon endoscope is to be stopped.

7. The system of claim 1, wherein the computing device is programmed by further executable instructions to:
determine, based at least partly on the procedure anomaly comprising an under-inflation anomaly, that inflation of the balloon is to be confirmed; and
display, on a user interface, a message indicating inflation of the balloon is to be confirmed.

8. The system of claim 1, wherein the computing device is programmed by further executable instructions to:
determine, based at least partly on the procedure anomaly comprising a deflection rate anomaly, that the remedial action comprises a reduction in a deflection rate of the balloon endoscope; and
display, on a user interface, a message indicating that the deflection rate of the balloon endoscope is to be reduced.

9. The system of claim 1, wherein the computing device is programmed by further executable instructions to:
determine, based at least partly on the procedure anomaly being one of an over-inflation anomaly or a low withdrawal speed anomaly, that the remedial action comprises a reduction in an inflation pressure of the balloon; and
generate a command for the control subsystem to reduce the inflation pressure of the balloon.

10. The system of claim 9, wherein to reduce the inflation pressure of the balloon, the command causes the control subsystem to switch the inflation pressure of the balloon to a lower pressure level.

11. The system of claim 9, wherein to reduce the inflation pressure of the balloon, the command causes the control subsystem to tune the inflation pressure of the balloon to a lower target pressure metric within a pressure range of a particular pressure level.

12. The system of claim 1, wherein the computing device is programmed by further executable instructions to:
determine, based at least partly on the procedure anomaly being one of an under-inflation anomaly or a high withdrawal speed anomaly, that the remedial action comprises an increase in an inflation pressure of the balloon; and
generate a command for the control subsystem to increase the inflation pressure of the balloon.

13. The system of claim 12, wherein to increase the inflation pressure of the balloon, the command causes the control subsystem to switch the inflation pressure of the balloon to a higher pressure level.

14. The system of claim 12, wherein to increase the inflation pressure of the balloon, the command causes the control subsystem to tune the inflation pressure of the balloon to a higher target pressure metric within a pressure range of a certain pressure level.

15. The system of claim 1, wherein the computing device is programmed by further executable instructions to:
determine, based at least partly on the procedure anomaly comprising a tissue-anomaly-related procedure anomaly, that the remedial action comprises an increase in an inflation pressure of the balloon to an anchoring pressure; and
generate a command for the control subsystem to increase the inflation pressure of the balloon to the anchoring pressure.

16. The system of claim 15, wherein the tissue-anomaly-related procedure anomaly comprises at least one of a tumor, a serious bleeding, a perforation or tear in the tissue, and a serious adverse condition of the tissue.

17. The system of claim 15, wherein to generate the command for the control subsystem, the computing device is programmed by further executable instructions to generate a command for a balloon inflation/deflation system.

18. The system of claim 1, wherein the computing device is programmed by further executable instructions to:
obtain a second image using the visualization element;
analyze the second image using the machine learning model to generate second classification output data;
determine, based at least partly on the second classification output data, that the second image corresponds to no procedure anomaly; and
cause presentation of a message regarding remediation of the procedure anomaly.

19. The system of claim 1, further comprising a training computing device comprising one or more processors and computer-readable memory, the training computing device programmed by executable instructions to at least:
obtain a plurality of images;
generate a plurality of training data images using the plurality of images, wherein images in a first subset of the plurality of training data images are associated with label data representing a negative classification for presence of the procedure anomaly, and wherein images in a second subset of the plurality of training data images are associated with label data representing a positive classification for presence of the procedure anomaly;
train the machine learning model using the plurality of training data images; and
distribute the machine learning model to one or more endoscope systems.

20. The system of claim 1, wherein the procedure anomaly is one of a plurality of procedure anomalies that the computing device is configured to detect using the machine learning model.

21-52. (canceled)
